(19) United States
(12) Patent Application Publication
(10) Pub. No.: US 2017/0304115 A1
(43) Pub. Date: Oct. 26, 2017

(54) DELIVERY DEVICE

(71) Applicant: Eyevation, LLC, Louisville, KY (US)

(72) Inventors: Norman Radtke, Louisville, KY (US); Dyson Hickingbotham, Wake Forest, NC (US)

(73) Assignee: Eyevation, LLC, Louisville, KY (US)

(21) Appl. No.: 15/525,936

(22) PCT Filed: Nov. 11, 2015
(86) PCT No.: PCT/US2015/060219

§ 371 Date: May 10, 2017

Related U.S. Application Data
(60) Provisional application No. 62/077,917, filed on Nov. 11, 2014.

(51) Int. Cl. A61F 9/00; A61M 5/31

(52) U.S. Cl. CPC A61F 9/0017; A61M 5/3137

(57) ABSTRACT

This invention provides a device for delivering medical implants, the device comprising a guide, a mandrel disposed internal to the guide, and a sleeve, wherein the sleeve is disposed external to the guide and is attached to the mandrel for movement therewith relative to the guide. Included among the various embodiments are devices configured for abutting a proximal surface against the palm of a user and presenting finger tabs for retraction of the guide relative to the mandrel. Optionally, a device of the present invention is a dual-mode device configured for being operated independently in each of an ejection mode and an injection mode. Optionally, a device of the present invention is configured for delivering an implant to the eye of a subject. Optionally, the eye implant is a retinal implant. Optionally, the implant comprises retinal cells.

### CROSS-REFERENCE TO RELATED APPLICATIONS

This application claims the benefit of U.S. Provisional Application No. 62/077917, filed Nov. 11, 2014.

### TECHNICAL FIELD

The present invention relates to delivery devices.

### BACKGROUND

Medical implantation procedures generally involve the delivery of a desired implant (e.g. tissue, drug, microchip, etc.) to a target site in a subject. One type of tool that has found use in certain procedures is the pushrod-barrel type device. The use of a pushrod-barrel type device for delivery typically involves the steps of loading a barrel with a desired substance, placing the distal end of the barrel at or near the target site in a subject, and discharging the substance from the barrel by sliding a pushrod through the barrel towards the distal end at the target site.

Difficulty can arise when the substance intended for extraction or delivery is fragile and/or when the target site is blocked or partially blocked by a delicate environment. Trauma to the surrounding environment or the substance itself can occur during the procedure in the event of off-target placement or unintentional movement of the device or extended duration of device use within the subject. Further tissue insult can ensue from the additional bulk of secondary devices such as cameras or fluid jets sometimes used to aid the procedure.

With the exception of the present inventor's prior devices, pushrod-barrel type devices have fallen short on a number of levels including their precision of device mobility and placement, ability to perform multiple functions, and simplicity of use in applications where the substance itself or the environment surrounding the target is delicate, for example, implantation/extraction procedures for nervous, cardiac, vascular, bone, and joint tissue.

Perhaps one of the more sensitive applications is the delivery or extraction of target substances to or from the eye, for example, implantation of retinal tissue into the subretinal space. Unfortunately, this type of surgery is rendered extremely difficult by both a delicate surrounding environment and a fragile target substance.

The subretinal space is the area between the retinal pigment epithelium (RPE) and the photoreceptors of the retina. The transparent, layered retina processes light images projected by the cornea and lens. The photoreceptor layer in the back of the retina transforms the light into electrical impulses. Other retinal layers transfer these impulses through the optic nerve to the brain which interprets the impulses into what we perceive as sight.

Normally, the photoreceptors are in close contact with the RPE. The RPE has many functions. It provides nutrition for the photoreceptors, and also removes waste products from the photoreceptors. In many diseases, the photoreceptors and retinal pigment epithelium degenerate. Such diseases include retinitis pigmentosa, dry age-related macular degeneration, Stargardt's disease, choroideremia, rod cone dystrophy, and Sjogren's reticular dystrophy. In a normal eye, there are no blood vessels in the subretinal space. However, in some retinal diseases, blood vessels and connective tissue can grow in this space. These abnormalities in the subretinal space under the retina can damage the retina in the back of the eye and can lead to blindness. Under certain disease conditions, the photoreceptors can detach very easily from the RPE. The photoreceptors will then degenerate, resulting in vision loss or blindness, while the other layers of the retina may remain functional. After removing the abnormal blood vessels, vision may be restored by replacing the diseased RPE and/or photoreceptors which may then integrate with the functional part of the retina.

Frequent causes of blindness are dry age-related macular degeneration and retinitis pigmentosa. The macula is located in the back of the eye in the central portion of the retina and is responsible for central vision. In subjects with dry age-related macular degeneration and retinitis pigmentosa, there is initially a dysfunction of the photoreceptors and/or RPE in the macular region. This results in impairment of central and/or peripheral vision. Age related macular degeneration is a disease that has been treated with piston-barrel type devices.

Retinitis pigmentosa is a term for genetically caused photoreceptor degeneration. In these subjects, the photoreceptors must be replaced. Again, such delivery can be accomplished by piston-barrel type devices.

Surgical correction of diseases in the subretinal space between the retina and the RPE is rendered extremely difficult by the environment in which the surgery must take place. Moreover, the surgical procedure disclosed herein to implant retinal tissue into the subretinal space of the eye is complicated by the fact that fetal retinal tissue is in the nature of a transparent gelatinous mass with two non-interdigitated layers, the RPE and the neural retina, consisting of neuroblastic cells and ganglion cells, and therefore extremely fragile.

Difficulties of implanting retinal tissue have been previously noted, as discussed in U.S. Pat. No. 5,941,250; U.S. Pat. No. 6,159,218; and U.S. Pat. No. 6,156,042, which describe delivery devices for implanting retinal tissue into the subretinal space of the eye. These devices are of the pushrod-barrel type. A tubular nozzle is telescoped over the distal end of the mandrel sleeve and is longitudinally slidable over the mandrel and onto the mandrel sleeve to eject the loaded retinal tissue. In the loaded position, the nozzle is positioned at the far distal end of the sleeve, but biased to slide back over the sleeve by a flat spring toggle having its distal end connected to the nozzle and its proximal end connected to the handpiece. However, none of U.S. Pat. No. 5,941,250; U.S. Pat. No. 6,159,218; and U.S. Pat. No. 6,156,042 teach a device with sensitive, error-reducing delivery control, a device having a nozzle that enables precise control of placement, or a device having a multifunctional mandrel.

U.S. Pat. No. 7,468,065 to Weber et al. describes a device for delivery of ocular implants, but like other devices discussed above, this device fails to meet the needs of applications involving fragile substances to be delivered or extracted, or where the target site is blocked by a delicate environment.

WO 2011/084550 to Hickingbotham et al. describes a device for delivery of ocular implants. However, it does not teach a device having a sleeve that slides about a guide with the advantages taught herein. Further, it also fails to teach a device configured for abutting against a user's palm while presenting finger tabs on the guide.

U.S. Pat. No. 4,994,028 to Leonard et al. describes a device for subcutaneous implantation of solid implants. However, it does not teach a device configured for abutting against a user's palm while presenting finger tabs on the guide. Further, the device fails meet the needs of applications involving fragile substances to be delivered or extracted, or where the target site is blocked by a delicate environment.

What are needed in the art are high-precision devices that are capable of rapid, non-invasive, delivery and/or extraction, yet simple to use and less prone to user error.

### SUMMARY OF THE INVENTION

The invention provides delivery devices, products comprising the delivery devices, kits comprising the delivery devices, methods of using the delivery devices, and methods of manufacturing the delivery devices.

A first aspect of the invention provides a delivery device comprising a discharge unit and a discharge unit guide, wherein:
a. the guide comprises an open end and can be loaded with an implant;
b. the discharge unit comprises:
   i. a mandrel disposed internally to the guide and configured to move relative to the guide to discharge a loaded implant from the open end of the guide; and
   ii. a sleeve disposed externally to the guide and attached to the mandrel for movement therewith relative to the guide.

The delivery device optionally comprises one or more features selected from:
a. at least one finger tab;
b. a device structured for abutting a proximal surface against the palm of a user's while operating the device to discharge an implant;
c. a device structured for providing the user with at least two modes of implant discharge—an injection mode and an ejection mode;
d. a sleeve comprising a lip and/or a cavity;
e. a rotation limiter for limiting rotation of the mandrel relative to the guide;
f. a retraction limiter for limiting retraction of the mandrel relative to the guide;
g. a curved nozzle;
h. a transparent nozzle;
i. an oblate nozzle;
j. a removable nozzle;
k. a tapered guide having segments with different diameters;
l. a mandrel comprising a flexible distal segment;
m. a mandrel comprising a rigid proximal segment;
n. a mandrel comprising a plunger head disposed proximally of the distal end, e.g. intermediate of a flexible distal segment and a rigid proximal segment;
o. is formed from a syringe barrel, a syringe needle, and/or a syringe plunger;
p. a side port in the guide for loading of an implant; and
q. a mandrel comprising a laterally deformable distal end.

Optionally, the device comprises at least one finger tab extending laterally from the guide, e.g. extending laterally from the guide in opposing directions. Optionally, the at least one finger tab is disposed distally of the sleeve. Optionally, the at least one finger tab comprises a distal surface configured for interfacing at least one finger of the user, e.g. interfacing two fingers situated about opposing surfaces of the guide. Optionally, when the device is in the loaded state, the at least one finger tab is offset from a proximal surface of the discharge unit configured for abutting against a palm of a user's hand (‘butt end’) such that the butt end can be abutted against the palm of a user's hand while the user's at least one finger interfaces with the distal surface of the at least one finger tab. Optionally, device is configured for providing the user with at least two modes of implant discharge—an injection mode and an ejection mode, wherein the injection mode comprises stabilizing the at least one finger tab and advancing the discharge unit (e.g. by moving a butt end of the discharge unit), and wherein the ejection mode comprises stabilizing the discharge unit and retracting the guide by moving the at least one finger tab.

Optionally, the discharge unit comprises a proximal surface configured for interfacing (e.g. abutting against) a palm of a user's hand (‘butt end’). Optionally, the butt end comprises a convex surface. Optionally, the proximal surface is disposed about the proximal end of the discharge unit (e.g. on the proximal end of the mandrel or the sleeve).

Optionally, the sleeve comprises a lip, a cavity, or both, e.g. disposed about a lateral surface of the sleeve. Optionally, the sleeve comprises both a lip and a cavity, wherein the cavity is in the lip. Optionally, the lip comprises proximal taper, a distal taper, or both. Optionally, the lip and/or the cavity are configured for interfacing a finger of a hand of the user. Optionally, the lip and/or the cavity are configured for interfacing a thumb of the hand of a user (e.g. when the palm of the user's hand interfaces an optional butt end of the discharge unit and/or when at least one finger other than the thumb interfaces an optional finger tab of the guide.

Optionally, the device comprises a mechanism for limiting rotation of the mandrel relative to the guide (‘rotation limiter’), e.g. and comprises an oblate nozzle and/or a mandrel with a wide tip. Optionally, rotation limiter is provided by a first member comprised by the guide and a second member comprised by the discharge unit, wherein the first member and second member are disposed circumferentially of each other and configured to move longitudinally relative to each other. Optionally, the first member is a pin comprised by the guide and the second member is a pin track comprised by the sleeve about which the pin moves longitudinally, optionally wherein the pin is recessed in the sleeve. Optionally, the pin track is a slot or a groove in the sleeve.

Optionally, the device further comprises a mechanism for limiting retraction of the mandrel relative to the guide (‘retraction limiter’). The retraction limiter can comprises, e.g. a tether connecting the guide and the discharge unit that prevents retraction beyond a given distance.

Optionally, the guide comprises a nozzle and the distal end. Optionally, the nozzle is curved. Optionally, the nozzle is transparent. Optionally, the nozzle is oblate. Optionally, the nozzle is flexible. Optionally, the guide comprises a nozzle and a segment proximal of the nozzle (‘body’) and the nozzle is removable from the body.

Optionally, the mandrel comprising a flexible segment at the distal end (‘flexible distal segment’). Optionally, the mandrel comprises a rigid segment proximal of the flexible distal segment. Optionally, the rigid proximal segment is a syringe plunger arm.

Optionally, the guide comprises a body and a segment distal of the body (‘distal segment’), wherein the distal guide segment is removable from the body (e.g. comprises a coupler such as a luer lock). Optionally, the body has greater diameter than the distal guide segment. Optionally, the body comprises at least one finger tab. Optionally, the distal segment comprises a nozzle (e.g. an oblate nozzle) and a segment proximal of the nozzle (e.g. a segment with a circular cross-section), wherein the segment proximal of the nozzle has a diameter that is less than that of the nozzle. Optionally, the segment proximal of the nozzle is formed from a syringe needle. Optionally, the body is formed from a syringe barrel.

Optionally, the guide comprises a side port. Optionally, the guide is a multilayer conformation having a plurality of concentric tubes and the side port passes through the plurality of concentric tubes.

Optionally, the mandrel comprising a plunger head disposed proximally of the distal end of the mandrel, e.g. intermediate of a flexible distal segment and a rigid proximal segment. Optionally, the plunger head is configured to impart suction and/or expulsion at the open end of the guide by pumping fluid. Optionally, the plunger head is a syringe plunger head (e.g. attached to a syringe plunger arm).

Optionally, the mandrel comprises a laterally deformable member at the distal end. Optionally, such a device comprising such a mandrel comprises a tapered guide having a plurality of segments with different diameters.

A second aspect of the invention provides a product comprising a delivery device of the invention (e.g. any taught above or hereinafter), an implant loaded in the device, and a sealed package comprising the device. Optionally, the sealed package is sterile. Optionally, the implant is comprises an ocular plant, retinal cells (e.g. retinal epithelial cells), a biodegradable fibrotic membrane, an SU-8 scaffold, an epoxy resin, an epoxy based viscous polymer, or a micropatterned implant.

A third aspect of the invention provides a kit comprising a device of the invention, and a plurality of alternate removable nozzles.

A fourth aspect of the invention provides a method of using a device of the invention (e.g. any taught above or hereinafter) for delivering an implant to a patient. Optionally, the method comprises delivering an implant (e.g. cells) to the subretinal space of a patient's eye. Optionally, the method comprises steps of loading the implant (e.g. cells) into the guide, inserting the guide through the surface of the eye, orienting the open end of the guide in proximity to the retina, sliding the open end of the guide under the retina and into the subretinal space, and discharging the implant by moving the mandrel through the guide towards the open distal end.

A fifth aspect of the invention provides a method of manufacturing a delivery device of the invention (e.g. any taught above or hereinafter). Optionally, the method comprises the steps of:
a. providing a syringe barrel;
b. providing a syringe needle;
c. providing a syringe plunger;
d. providing a nozzle;
e. providing a sleeve configured to accept the syringe barrel;
f. providing an elongated member configured for insertion through the syringe needle and the nozzle (‘push rod’);
g. inserting the syringe plunger in the sleeve;
h. attaching the plunger to the sleeve;
i. attaching the push rod to the syringe plunger;
j. wherein the push rod is attached to the syringe plunger arm at a location distal of the attachment point of the sleeve and the plunger;
k. attaching the nozzle to the syringe needle;
l. attaching the syringe needle to the syringe barrel; and
m. inserting the mandrel into the guide.

The invention also provides a device made by a method of manufacturing of the invention.

### BRIEF DESCRIPTION OF THE DRAWINGS

FIG. 1 depicts a delivery device of the invention.

FIG. 2A-FIG. 2D depict sectioned views of a delivery device of the invention.

FIG. 3 depicts a front perspective view of a delivery device of the invention.

FIG. 4A depicts an exploded view of a delivery device of the invention.

FIG. 4B depicts an exploded view of a guide useful in a delivery device of the invention.

FIG. 4C depicts an exploded view of a discharge unit useful in a delivery device of the invention.

FIG. 5 depicts a front perspective view of delivery device of the invention.

FIG. 6A and FIG. 6B depict sectioned views of a delivery device of the invention in a discharged state and loaded state, respectively.

FIG. 7 depicts a rear perspective view of a delivery device of the invention.

FIG. 8A-C depict a top view, an elevation view, and a perspective view, respectively, of a curved nozzle useful in the present invention.

FIG. 9A-C depict a top view, an elevation view, and a perspective view, respectively, of a straight nozzle useful in the present invention.

FIG. 10 depicts a front view of a nozzle useful in the present invention with the nozzle heights and widths labeled.

FIG. 11 depicts a manner of holding a device of the present invention useful, e.g. for an ejection mode of discharge.

FIG. 12A and B depict a manner of holding a device of the present invention useful, e.g. for an injection mode of discharge.

FIG. 13 depicts a manner of holding a device of the present invention useful, e.g. for an injection mode of discharge.

FIG. 14 depicts a manner of holding a device of the present invention useful for either injection or ejection modes of discharge.

FIG. 15 depicts a manner of holding a device of the present invention useful, e.g. for an ejection mode of discharge.

FIG. 16 depicts a manner of holding a device of the present invention useful, e.g. for an ejection mode of discharge.

FIG. 17 depicts a delivery device of the invention.

FIG. 18A and FIG. 18B depict a front (distal) elevation view and a perspective view, respectively, of a sleeve useful in the present invention.

FIG. 19 and FIG. 19B depict a front (distal) elevation view and a perspective view, respectively, of a sleeve useful in the present invention.

FIG. 20A, FIG. 20B, and FIG. 20C depict optional dimensions of a delivery device of the invention.

FIG. 21A depicts a guide segment useful in the present invention. FIG. 21B depicts an exploded view thereof.

FIG. 22A depicts a mandrel segment useful in the present invention. FIG. 22B depicts an exploded view thereof.

FIG. 23A depicts a first perspective view of a delivery device of the invention. FIG. 23B depicts a second perspective view of the delivery device. FIG. 23C depicts an exploded top view of the delivery device. FIG. 23D depicts an exploded elevation view. FIG. 23E depicts a partial view of the delivery device detailing the distal end of the mandrel. FIG. 23E is a portion of the view depicted in FIG. 23D enlarged for magnification purposes. FIG. 23E corresponds to the portion of FIG. 23D circled with a dashed line and labeled “23E”.

FIG. 24 depicts two states of lateral expansion of a useful distal member of a mandrel of a delivery device of the present invention.

FIG. 25 depicts a portion of a nozzle and a distal member of a mandrel traveling through he nozzle. FIG. 25 depicts a distal member 401 in two alternate positions. The retracted position of the member 401 is depicted in solid lines The advanced position of the member 401 is depicted in dashed lines.

### DETAILED DESCRIPTION OF THE INVENTION

As used here, the following definitions and abbreviations apply.

“Examplary” (or “e.g.” or “by example”) means a non-limiting example.

“Cross-section” means lateral cross-section.

“Diameter” means the distance across a lateral dimension of a component. The diameter of any shape can be measured. For example, the shape can be circular (i.e. have a circular cross-section) or non-circular, cylindrical or non-cylindrical, symmetrical (i.e. have a symmetrical cross section) or non-symmetrical.

“Discharged state” means the state of a delivery device in which the implant has been fully discharged from the guide. For example, the discharged state can be the state in which the distal end of the mandrel is flush with the distal end of the guide.

“Distal” means situated away from the user. For example, the distal end of the guide is the end which contacts or is placed about the subject or target site therein.

“Distal surface” means a surface facing the distal end.

“FEP” means fluorinated ethylene propylene

“Loaded state” means the state of a delivery device in which the mandrel is retracted relative to the guide and an implant, if present, is retained in the guide.

“Lateral” means of or relating to a dimension that is perpendicular to a longitudinal dimension.

“Lateral surface” means a surface that is offset laterally from the mandrel.

“Longitudinal” means of or relating to a dimension or path along which longitudinal movement occurs.

“Longitudinal movement” means movement in a direction in which the mandrel can move relative to the guide to discharge an implant from an open end of the guide or to intake an implant at the open end of the guide. Longitudinal movement can be advancement or retraction. For example, discharge of a loaded implant can occur via advancement of the mandrel or retraction of the guide.

“Luminal diameter” means the diameter of the inner surface of a hollow structure. For example, the luminal diameter of a tube is the distance between opposing luminal surfaces.

“Opposing” means facing or extending in different directions. For example, the different directions can be 90° to 180° apart. Optionally, opposing surfaces and/or opposing directions are 130° to 180° apart.

“Proximal” means situated away from the distal end of the device longitudinally. For example, in some embodiments, the proximal end of the device is can be abutted against the user's palm.

“Proximal surface” means a surface facing away from the distal end of the device.

“RPE” means retinal pigment epithelium.

“Wide” means a component has two lateral dimensions that are perpendicular to each other and substantially different measurements. For example, a nozzle having a cross-section with a width two times greater than the height is a wide nozzle. Optionally, a first of the dimensions is at least two times (e.g. at least three times) greater than a second of the dimensions.

### Device Overview

The invention provides a delivery device comprising a discharge unit and a discharge unit guide (‘guide’). The guide comprises an open end and can be loaded with an implant, e.g. loaded via the open end or loaded via an optional side port.

The discharge unit comprises a mandrel and a sleeve. The mandrel is disposed internally to the guide, and optionally internal to the sleeve, and is configured to move longitudinally relative to the guide to discharge a loaded implant from the open end. The sleeve is disposed externally to the guide and attached to the mandrel (e.g. the proximal end of the mandrel) for movement therewith relative to the guide. For example, the mandrel is optionally fixed in position relative to the sleeve such that the mandrel and sleeve move as a single unit.

In one illustrative embodiment of invention, the mandrel comprises a pushrod and the guide is configured as a tube through which the pushrod slides to discharge an implant from an open distal end of the tube. The sleeve is attached to the proximal end of the mandrel, or pushrod thereof, and slides over the proximal end of guide, providing one or more surfaces for contacting a user (e.g. a finger of a user).

An implant loaded in the guide can be discharged, e.g. as a result of force applied directly from the mandrel, i.e. there is a direct interface of the implant with the distal end of the mandrel. Alternatively, the implant can be discharged as a result of an indirect force applied from the mandrel such as by fluid pressure created in the guide by longitudinal movement of the mandrel (e.g. fluid pressure imparted by a plunger head).

### Discharge Unit Guide

A delivery device of the invention comprises a discharge unit guide (‘guide’) through which a mandrel can move longitudinally. The guide comprises at least two functional segments: a nozzle and a body. The nozzle is located at the distal end of the guide and is configured for insertion into a subject (e.g. a human subject) and the implant can be discharged from the nozzle to deliver the implant to a target site in the subject. The body is proximal of the nozzle and is optionally configured for slidably receiving the sleeve of the discharge unit.

The guide can be provided in any shape or configuration as long as an implant loaded in the guide (e.g. nozzle thereof) and can by discharged by relative longitudinal movement of a mandrel towards the distal end of the guide (‘mandrel advance’). For example, the guide can be provided as a cannula or a track, wherein the mandrel moves longitudinally about (e.g. through) the guide.

Optionally, the guide comprises two or more members coupled together to form a functional guide. Alternatively, the guide is formed as a single member, for example, where the body and the nozzle are formed together in a mold. The body and nozzle can have different lateral cross sections or can have the same or similar lateral cross sections.

### Nozzle

The nozzle of a delivery device guide has an open distal end and is configured for insertion in a subject. The nozzle is optionally configured to hold any implant therein prior to discharge of the implant.

The nozzle can be any shape or size and can be constructed from any materials.

Optionally, the nozzle is a wide (e.g. an oblate) nozzle.

Optionally, the nozzle is a curved nozzle or a straight nozzle.

Optionally, the nozzle is rigid or flexible. Optionally, the nozzle has greater flexibility than the distal end of the mandrel (e.g. such that the distal end of the mandrel conforms and becomes curved advancing traveling through a curved nozzle).

Optionally, the nozzle is removable from the body of the guide (‘removable nozzle’).

Optionally, the nozzle is transparent.

Optionally, the nozzle is transparent and flexible.

Optionally, the nozzle is plastic, such as an elastic plastic or a thermoelastic polymer (e.g. fluorinated ethylene propylene (‘FEP’).

Optionally, the nozzle is configured (e.g. shaped) to deliver a particular implant. Optionally, the nozzle is configured to match-fit the implant. Optionally, the implant is an ocular implant. Optionally, the implant comprises retinal tissue. Optionally, the implant is a sheet, for example, a sheet of RPE, a nanoplate, a membrane (e.g. biodegradable fibrotic membrane) Optionally, the implant is a sheet comprising retinal tissue or stem cells. Optionally, the nozzle is a wide nozzle configured to match fit a sheet.

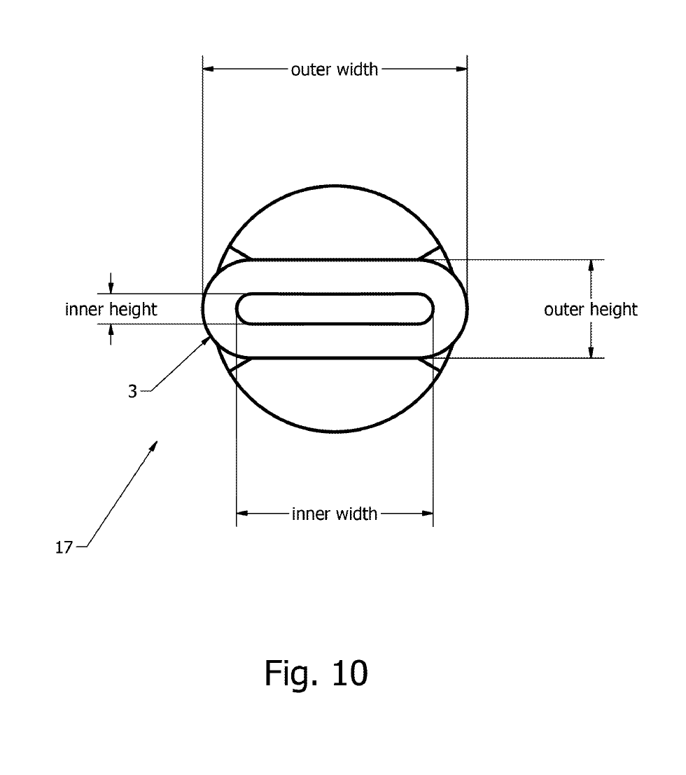

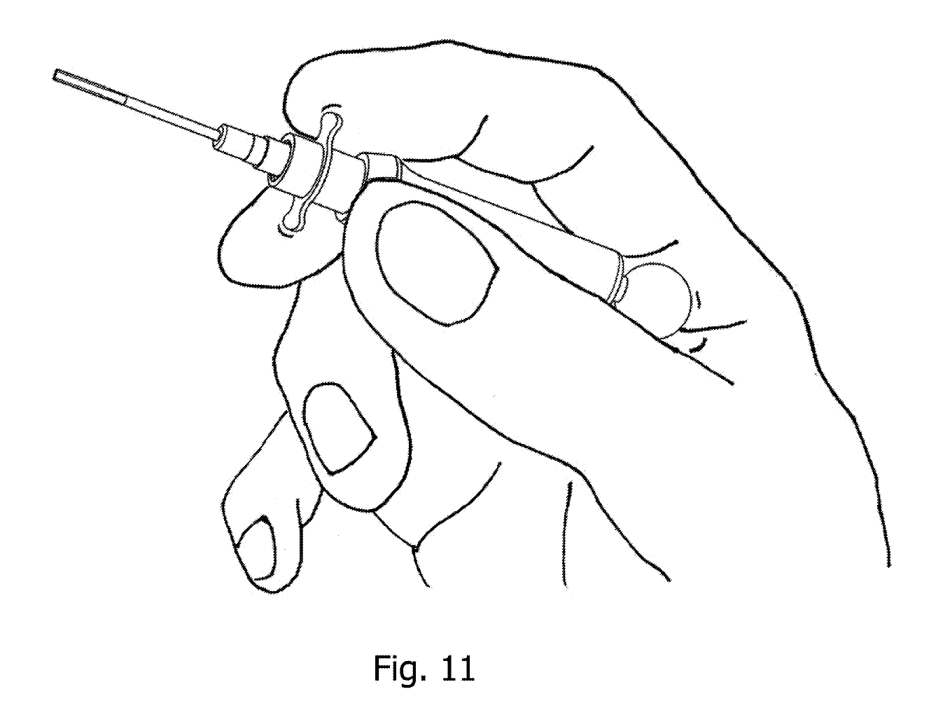

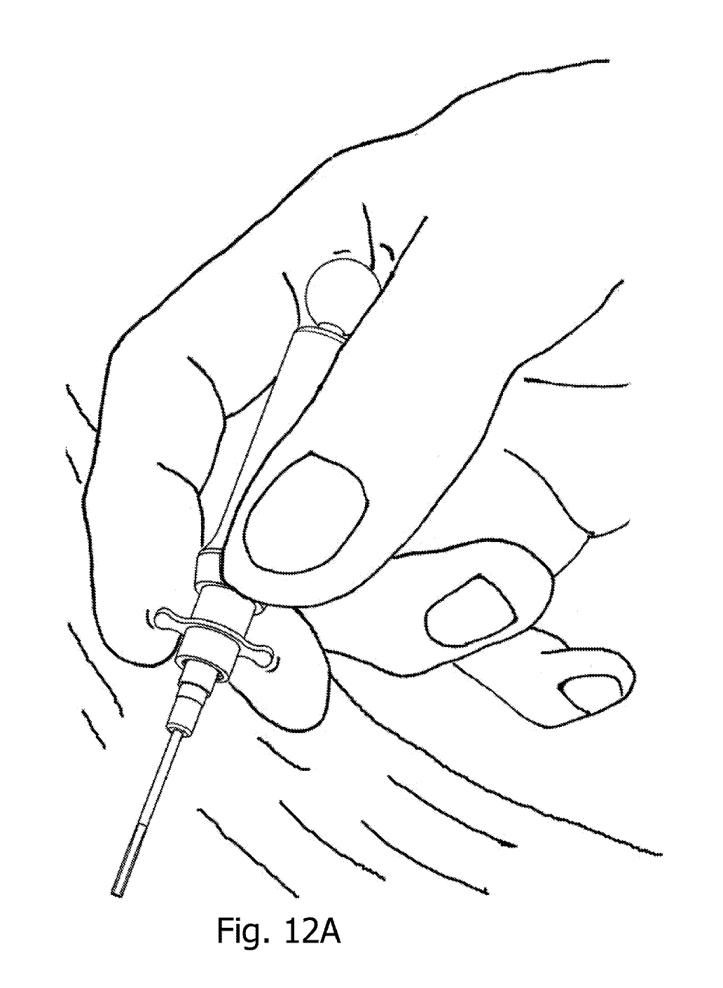

Optionally, the nozzle is a wide nozzle. An example of a wide nozzle is illustrated in FIG. 10. The wide nozzle can be, for example, any nozzle having an aspect ratio (e.g. width:height of the inner diameter or outer diameter) substantially greater than 1:1 or a nozzle in which a lateral cross section of the nozzle (i.e. the cross section taken perpendicular to the longitudinal direction of travel of the mandrel) has perpendicular dimensions (e.g. height and width) that are substantially different. Examples of a wide nozzle include an oblate nozzle, a rectangular nozzle (e.g. a rectangle with curved edges and/or corners). Optionally, the wide nozzle is wide at the distal end and cylindrical (or having a circular lateral cross section) and the proximal end, for example, for attachment to a cylindrical segment of the guide proximal of the nozzle. Optionally, the wide nozzle has an aspect ratio of at least about 3:2 (e.g. at least 2:1 or at least 5:2), and optionally less than about 20:1. Optionally, the wide nozzle has an aspect ratio of at least about 5:1 (‘ultra-wide nozzle’), e.g. at least about 8:1 (e.g. about 5:1 to about 15:1). Optionally, the wide nozzle (e.g. ultra-wide nozzle) has a wall thickness of less than half of the height.

Optionally, the nozzle is configured to match fit an implant. A nozzle that is configured to match fit an implant contacts the implant about a plurality of surfaces (e.g. opposite surfaces) of the implant when loaded in the nozzle or when being discharged from the nozzle. Optionally, the implant makes at least three points of contact with the nozzle, a first point and a second point on opposite surfaces of the implant, and a third point located between the first point and the second point, optionally wherein third point is equidistant (e.g. measured by degrees of rotation or by circumference) from the first point and the second point. Optionally, the implant makes a fourth point of contact with the nozzle, wherein the fourth point is on an opposite surface relative to the third point, and optionally wherein a first imaginary segment is perpendicular to a second imaginary segment, wherein the first imaginary segment connects the first and second points and the second imaginary segment connects the third and fourth points. Optionally, the majority (e.g. substantially all) of the implant surface that is surrounded by the nozzle is contacted by the luminal wall of the nozzle.

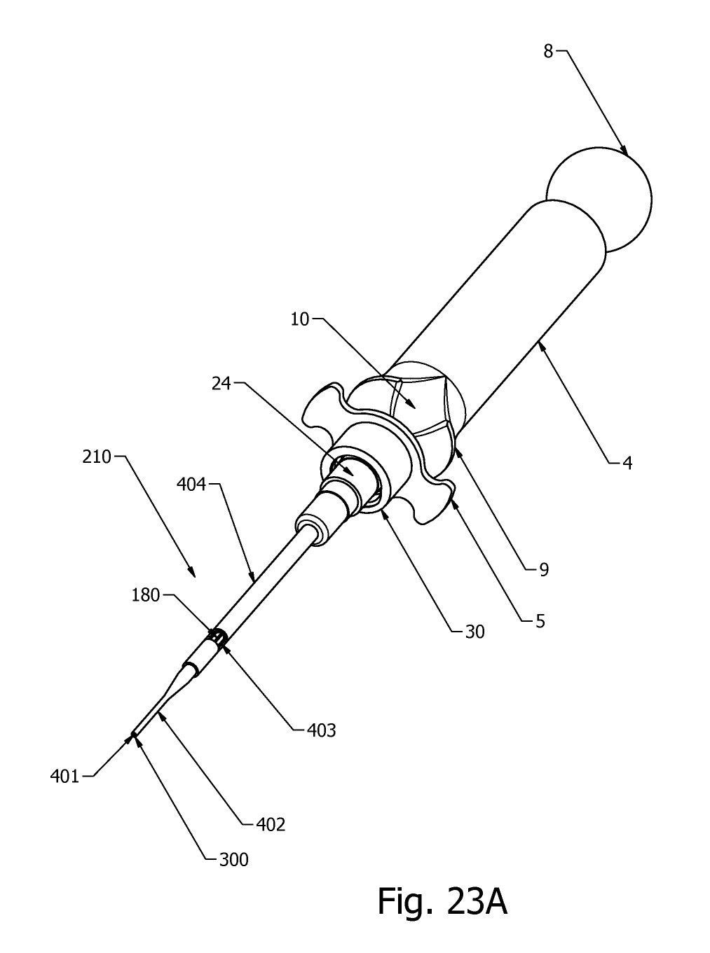

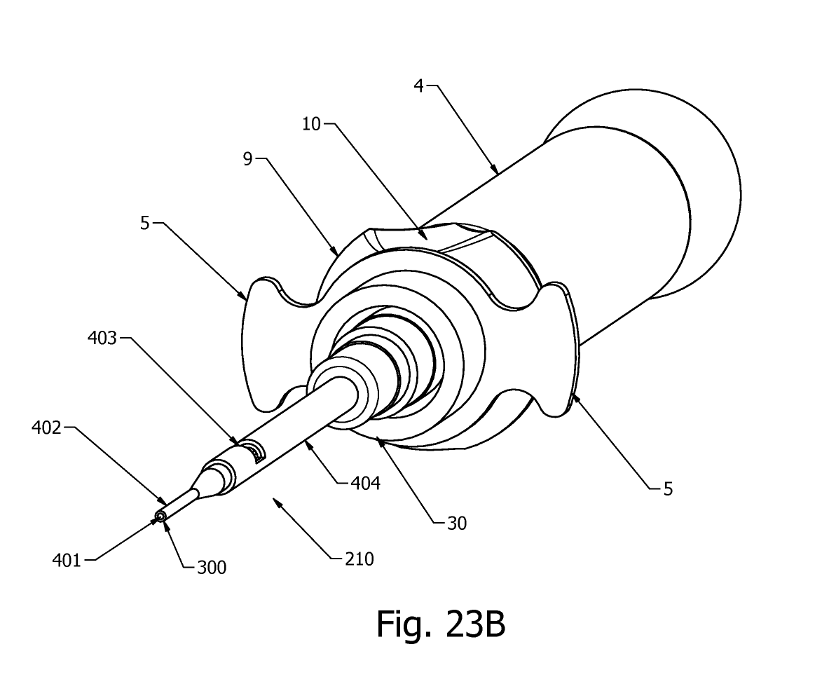

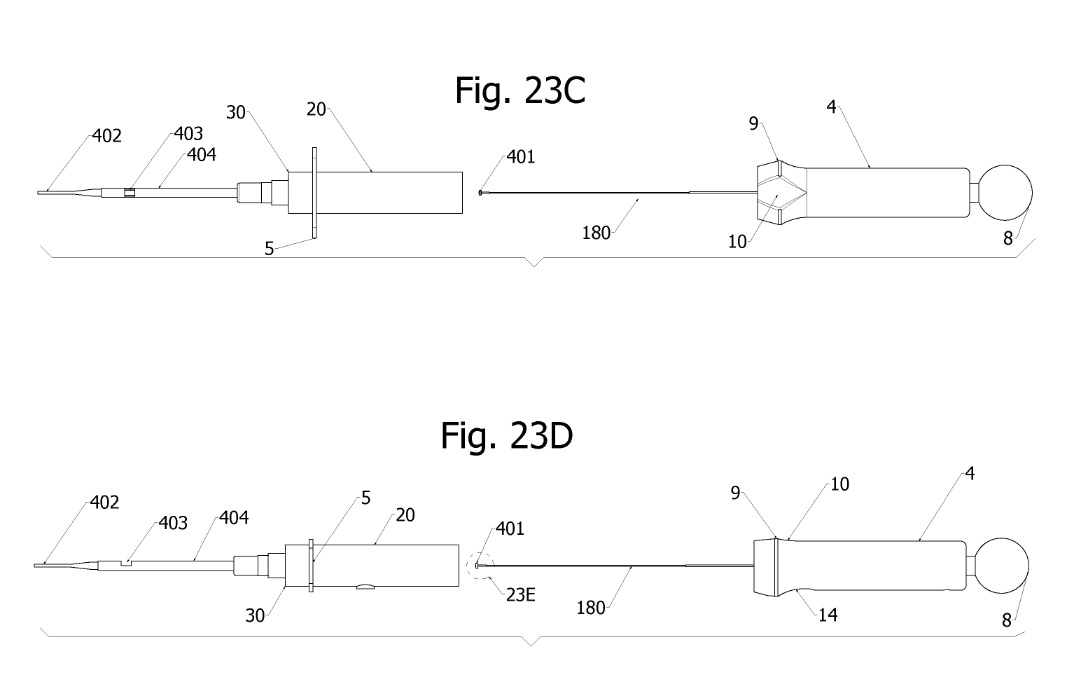

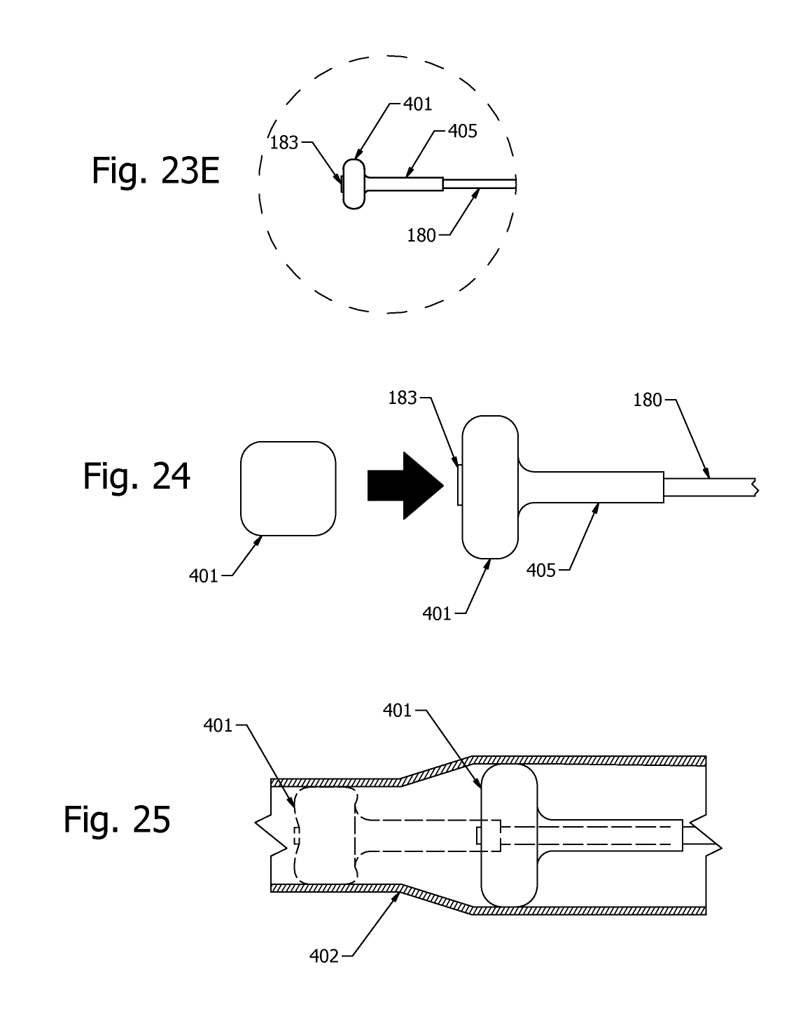

Optionally, the distal end of the nozzle (e.g. a wide nozzle) has the greatest lateral dimension (i.e. dimension perpendicular to the direction of longitudinal movement of the mandrel) of the portion of the guide that is inserted into a subject. Alternatively, the distal end of the nozzle (e.g. nozzle of a tapered guide) can have has smallest lateral dimension of the guide, e.g. as depicted in FIG. 23C.

Optionally, the nozzle is a removable nozzle. A removable nozzle is any nozzle that can be attached and detached from the body of the guide. A device having such a nozzle may be used with removable nozzles of different shapes and/or sizes in order to use the same device for different applications (e.g. delivery of different implants or used with different subjects or delivery sites). A device with a removable nozzle comprises a coupler for attaching and detaching the nozzle from the body. The nozzle and the body can be directly coupled to each other such that there are no intermediate segments between the body and the nozzle or indirectly coupled to each other such that there is at least one intermediate segment disposed between the coupler and the nozzle. Optionally, the coupler is a luer lock coupler or a slip tip coupler. Other useful nozzle couplers include those taught in WO/2011/084550.

Optionally, the nozzle is a nozzle having the dimensions of any of the examples listed in Table 1. Optionally, the dimensions are the dimensions at the open end of the nozzle. Optionally, the nozzle is comprised by a device used to deliver an implant to the eye of an optional subject listed in Table 1.

TABLE 1
Nozzle Dimensions

| | inner height | | inner width | outer height | outer width | Optional subject |
|---|---|---|---|---|---|---|
| Example-wide nozzle | 0.5 | mm | 1.32 mm | 0.58 mm | 1.4 mm | |
| Example-ultra wide nozzle | 87 | um | 1 mm | 163 um | 1.08 mm | |
| Example-circular nozzle | 1 | mm | 1 mm | 1.2 mm | 1.2 mm | |
| Example-wide nozzle | 0.5 mm-1.5 mm | | > inner height | > inner height | > outer height | human |
| Example-ultra wide nozzle | 0.05 mm-0.15 mm | | >5× inner height | > inner height | > outer height | human |
| Example-circular nozzle | 0.5 mm-1.5 mm | | = outer height | > inner height | = outer height | human |
| Example-wide nozzle | <1 | mm | > inner height | > inner height | > outer height | Rodent or human |
| Example-ultra wide nozzle | <.5 | mm | >5x inner height | > inner height | > outer height | Rodent or human |
| Example-circular nozzle | <1 | mm | = outer height | > inner height | = outer height | Rodent or human |

Optionally, the nozzle is substantially cylindrical or has a circular cross section at the open end. Optionally, such a nozzle is useful for insertion through the cornea. Such a nozzle is useful, for example, for being loaded with an implant (e.g. a sheet, such as a membrane, comprising a therapeutic substance such as RPE cells) folded or rolled, such as a 0.5 mm disc of 200 microns rolled and loaded in the nozzle. Optionally, the nozzle has an inner diameter of about 0.05 mm to about 0.3 mm, e.g. about 0.05 mm to about 0.2 mm or about the size of a 35 gauge needle to a 25 gauge needle (e.g. the size of a 33 gauge needle or within 3 gauge sizes thereof).

### Body

The body of the discharge unit guide is proximal of the nozzle and configured for receiving a sleeve and sliding longitudinally about the mandrel and/or the sleeve.

The body can, for example, be configured to make sliding contact with both the mandrel and the sleeve during travel of mandrel through the guide. Optionally, the barrel is configured with an inner diameter substantially the same as the outer diameter of at least a portion of the mandrel (e.g. plunger head) that slides through the body and comprises an outer diameter substantially the same as the inner diameter of the sleeve through which the body slides.

Optionally, the body has a lateral dimension substantially greater than that of the nozzle. For example, the lateral extension of the body lumen can be greater than that of the nozzle lumen and/or the lateral extension of the body outer surface can be greater than that of the nozzle outer surface.

Optionally, the body comprises a cannula (e.g. having a cylindrical lumen) through which at least a portion of the mandrel travels.

Optionally, the body comprises a cylindrical outer surface, e.g. about which a sleeve slides. Alternatively, the body comprises an outer surface in the shape of any prism (e.g. cylindrical prism or polygonal prism).

Optionally, the body is provided by a syringe barrel, e.g. an off-the-shelf syringe barrel.

Optionally, the delivery device comprises at least one finger tab extending from the body. Optionally, the at least one finger tab extends from the body at a location distal of the sleeve, e.g. distal of the sleeve at all times during use, when in the loaded state, or when in the discharged state.

Finger tabs

A delivery device of the invention optionally comprises at least one finger tab extending laterally from the guide. Optionally, the at least one finger tab extending laterally from the guide in opposing directions.

Optionally, the at least one finger tab extends laterally from the body of the guide.

Optionally, the at least one finger tab is disposed distally of the sleeve.

Optionally, the sleeve comprises a lip and/or a cavity and the at least one finger tab is disposed distally of the lip and/or a cavity.

Optionally, the at least one finger tab is a single finger tab extending laterally from the guide in opposing directions (e.g. a finger tab ring surrounding the guide) or is a plurality of finger tabs extending from the guide in opposing directions (e.g. a first finger tab extending form one surface of the guide and a second finger tab extending from an opposing surface of the guide).

Optionally, the at least one finger tab comprises a distal surface configured for interfacing at least one finger of the user. Optionally, the distal surface is substantially perpendicular to the direction of longitudinal movement of the mandrel through the guide or is otherwise configured such that a normal force can be imparted by a substantial portion of a user's finger to induce longitudinal movement to retract the guide relative to the mandrel (e.g. as detailed in Example 1 and Example 2). This is in stark contrast to the thumb knob in the device described by U.S. Pat. No. 4,994,028 to Leonard et al., which does not provide such a distal surface for interfacing a substantial portion of a user's finger but instead presents a lateral surface for interfacing a user's finger. Optionally, the at least one finger tab comprises at least one distal surface configured for interfacing two fingers situated about opposing surfaces of the guide.

Optionally, the at least one finger tab comprises a distal surface, wherein when the device is in the loaded state, the at least one finger tab is offset from a butt end of the discharge unit such that the butt end can be abutted against the palm of a user's hand while the user's at least one finger interfaces with the distal surface of the at least one finger tab.

### Segmented Guide

A discharge unit guide optionally comprises a plurality of serially connected segments having different structures (‘segmented guide’). Optionally, at least two of the segments are removable from each other (e.g. connected by a coupler).

Optionally, the guide comprises a body and a segment distal of the body comprising the nozzle (‘distal guide segment’). Optionally, the body and the distal segment are connected to each other by a coupler (e.g. luer lock). Optionally, the distal segment comprises a nozzle (e.g. an oblate nozzle) and a segment proximal of the nozzle (e.g. a segment with a circular cross-section), wherein the segment proximal of the nozzle has a diameter that is less than that of the nozzle. Optionally, the segment proximal of the nozzle is formed from a syringe needle. Optionally, the body is formed from a syringe barrel.

Optionally, the guide comprises a distal segment and a body, wherein the distal segment is removable from the body (e.g. comprises a coupler such as a luer lock). Optionally, the body has a greater diameter than the distal guide segment. Optionally, the body comprises at least one finger tab. Optionally, the distal segment comprises a nozzle (e.g. an oblate nozzle) and a segment proximal of the nozzle (e.g. a segment with a circular cross-section), wherein the segment proximal of the nozzle has a diameter that is less than that of the nozzle. Optionally, the segment proximal of the nozzle is formed from a syringe needle. Optionally, the body is formed from a syringe barrel.

### Side Port

A discharge unit guide optionally comprises a port in the sidewall of the guide (‘side port’). Such a side port can be configured, for example, to provide access to load an implant into the guide when the mandrel is retracted to a position proximal of the side port. The side port can optionally be in any location distal of the body and proximal of the open distal end such that the side port is not coextensive with the open distal end. For example, the side port can be in a location of the guide that is not inserted into a subject.

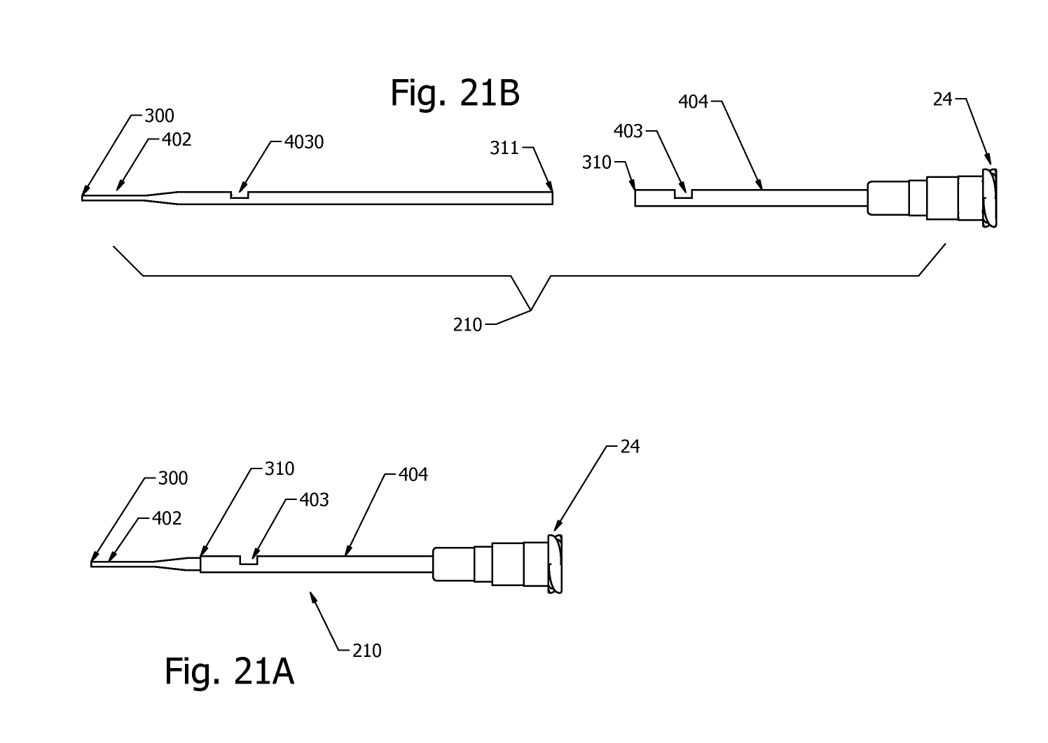

Optionally, the guide comprises a plurality of layers (e.g. bilayer) of tubes (e.g. concentric tubes) and a port extending through the sidewalls of each the tubes. Optionally, the plurality of layers comprise a first layer (e.g. first tube) comprising the nozzle and a second layer (e.g. second tube such as a segment proximal of the nozzle), wherein at least a portion of the first layer is inside or outside of the second layer. Optionally, the first layer is delicate and/or flexible relative to the second layer. Optionally, the second layer is thicker, stronger, and/or more rigid than the first layer. An example of a distal portion of such a guide, which comprises the nozzle, is depicted in FIGS. 21A and 21B.

Optionally, the guide comprises a nozzle and a segment proximal of the nozzle, wherein the side port is in the segment proximal of the nozzle. Such a segment proximal of the nozzle can be, for example, a rigid segment (e.g. steel such as stainless steel). Optionally, the nozzle in such a configuration is flexible and/or plastic (e.g. FEP). Optionally, the guide comprises a tubular member extending proximally from the nozzle, wherein the tubular member is at least partially disposed inside the segment proximal of the nozzle, wherein the proximal end of the tubular member is proximal of the side port, and wherein the side port passes through the side wall of both the segment proximal of the nozzle and the tubular member. Optionally, the tubular member is made from the same material as the nozzle and is seamlessly connected to the nozzle, e.g. formed together with the nozzle by molding or extruding. Optionally, the wherein guide tapers in diameter from the tubular member to the nozzle, e.g. to provide a smaller diameter nozzle for insertion into a subject.

### Multilayer Guide

Optionally, the guide comprises a plurality of layers (e.g. bilayer) of tubes (e.g. concentric tubes) and a port extending through the sidewalls of each the tubes. Optionally, the plurality of layers comprise a first layer (e.g. first tube) comprising the nozzle and a second layer (e.g. second tube such as a segment proximal of the nozzle), wherein at least a portion of the first layer is inside or outside of the second layer. Optionally, the first layer is delicate and/or flexible relative to the second layer. Optionally, the second layer is thicker, stronger, and/or more rigid than the first layer.

### Discharge Unit

A delivery device of the invention comprises a discharge unit comprising a mandrel and a sleeve. The mandrel disposed internally to the delivery unit guide and is configured to move relative to the guide to discharge a loaded implant from the guide. The sleeve is disposed externally to the guide and is attached to the mandrel for movement therewith relative to the guide.

### Mandrel

The mandrel is an elongated member that slides about the delivery unit guide and interfaces with an implant, when loaded in the guide, at the distal end of the mandrel. The mandrel may be constructed in any manner that induces longitudinal movement of the implant in the same direction as the mandrel when the mandrel moves longitudinally relative to the guide. Accordingly, a mandrel at least comprises a pushrod for discharging an implant.

Optionally, the mandrel comprises an elongated metal (e.g. steel) member such as a wire or an elongated plastic member. Optionally, the mandrel is autoclavable.

The mandrel can be flexible or rigid. Optionally, a portion of the mandrel is flexible (e.g. flexible pushrod) and a portion of the mandrel is rigid (rigid plunger arm). Optionally, the flexible portion is distal of the rigid portion.

Optionally, the mandrel comprises a pushrod and a segment proximal of the pushrod.

### Pushrod

A pushrod can be configured in any manner that interfaces with an implant when loaded in the guide.

Optionally, the pushrod is flexible, e.g. relative to the nozzle or an optional proximal mandrel segment. Optionally the pushrod is flexible such that it conforms as it travels through a curved nozzle.

Optionally, the mandrel is autoclavable.

Optionally, the distal end of mandrel is configured (e.g. shaped) to deliver a particular implant (e.g. a sheet or wide implant) and/or to travel through a particular nozzle. Optionally, the pushrod a wide distal end having greater lateral extension than a portion of the pushrod proximal of the distal end. For example, a pushrod can be obtained by providing a wire and looping one end of the wire to form a wide distal end. As another example, the pushrod can comprise, at the distal end, a laterally deformable member.

Optionally, the mandrel comprises, at its distal end, a laterally deformable member. Such a deformable member can be provided, for example, such that it can fill and conform to the luminal wall of the guide and/or be compressed by the luminal wall of the guide. Optionally, the laterally deformable member has a snug fit in the lumen of the guide. Optionally, the guide comprises a first segment having a first inner diameter and a nozzle comprising a second inner diameter that is less than the first inner diameter, and wherein the mandrel can be advanced from position that disposes the laterally deformable member in the first segment to a position that disposes the laterally formable member in the nozzle and wherein the laterally deformable member can be compressed by the side wall of the nozzle. Optionally, the laterally deformable member is laterally expanded by longitudinal compression, for example, sandwiched longitudinally between two fixed members such as a compression sleeve and a swaged distal end of the mandrel. Optionally, the laterally deformable member comprises silicone, rubber, foam, or any deformable material. Optionally, the material is elastic. Optionally, a laterally deformable member material is selected such that it can be laterally compressed by the guide or nozzle thereof, without damage or deformation of the guide.

### Proximal Mandrel Segment

A mandrel useful in the present invention optionally comprises a segment proximal of the pushrod (‘proximal mandrel segment’).

Optionally, the proximal segment is less flexible relative to the pushrod.

Optionally, the proximal segment has a greater diameter than the pushrod.

Optionally, the proximal mandrel segment comprises a plunger head. The plunger head can be, e.g. a soft member (e.g. made from rubber or a thermoplastic elastomer) configured with a snug, leak-free fit in the guide body such that it acts as a fluid pump when moved longitudinally through the guide body. Optionally, the plunger head is configured to impart suction and/or expulsion at the open end of the guide by pumping fluid. Optionally, the plunger head is a syringe plunger head (e.g. attached to a syringe plunger arm).

Optionally, the proximal segment is formed from a syringe plunger arm.

Optionally, the proximal segment is attached to the sleeve, e.g. at the proximal end of the mandrel.

### Sleeve

The sleeve is disposed externally to the guide and attached to the mandrel for movement therewith relative to the guide. Optionally, the luminal surface of the sleeve makes sliding contact with the outer surface of the body of the guide.

Optionally, the sleeve is configured such that the surface area of sliding contact between the body and the luminal surface of the sleeve increases as the sleeve is advanced relative to the body, e.g. as in the device detailed in Example 1 and Example 2.

Optionally, the lumen of the sleeve has a prism shape (e.g. a polygonal prism or a cylinder). Optionally, the lumen of the sleeve is substantially cylindrical.

Optionally, the sleeve comprises a finger grip, e.g. a lip, cavity, or friction pad.

Optionally, the luminal surface of the sleeve makes sliding contact with the outer surface of the body of the guide, e.g. as detailed in Example 1 and Example 2. Optionally, the sleeve is an elongated sleeve (i.e. having a longitudinal length substantially greater (e.g. at least two or three times greater) than the diameter of the luminal surface). Optionally, the surface area that makes sliding contact between the sleeve and the body is substantially greater (e.g. at least two or three times greater) than the distance of longitudinal movement required to discharge a loaded implant. The distance of longitudinal movement can optionally be governed, e.g. by the nozzle length (e.g. the length of a wide portion thereof), a retraction limiter, or the length of the implant.

Optionally, the diameter of luminal surface of the sleeve which makes sliding contact with the body is substantially greater (e.g. at least 5 times greater) than the luminal diameter of the nozzle, e.g. as detailed in Example 1 and Example 2.

Optionally, the diameter of the outer surface of the sleeve is less than about 4 cm, for example, less than about 3 cm, less than about 2 cm, or less than about 1.5 cm. Optionally, the diameter of the outer surface of the sleeve is greater than about 0.5 cm, for example, greater than about 1 cm. Optionally, the diameter of the outer surface of the sleeve is about 0.5 cm to about 2 cm.

Independently or combined, the length and diameter of the sliding contact surface area between the sleeve and the body provides an extremely smooth and guided longitudinal movement of the discharge unit relative to the guide, e.g. as detailed in Example 1 and Example 2.

The sleeve can be any longitudinal length. Optionally, the device comprises a sleeve and a body, wherein the length of contact between the sleeve and the body is at least about 2 cm, e.g. at least about 3 cm, at least about 4 cm, about 2 cm to about 9 cm, about 2 cm to about 8 cm, or about 3 cm to about 7 cm.

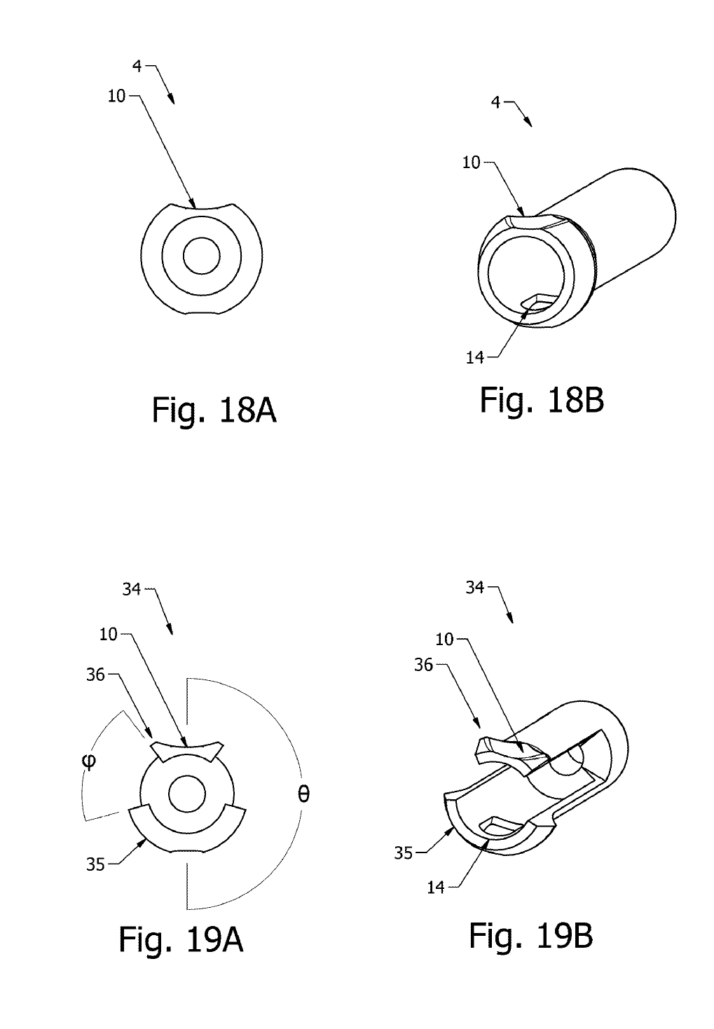

Optionally, the sleeve surrounds the body. Optionally, the sleeve is configured to surround the body such that the sleeve provides at least two contact points (i.e. points on the sleeve surface) spaced circumferentially by at least 90°, for example, by at least 120°, by at least 150°, or by 180° (e.g. as depicted by angle theta θ in FIG. 18A,B and FIG. 19A,B). Optionally, the sleeve is configured to fully surround the body, e.g. as depicted in FIG. 18A and FIG. 18B. Alternatively, the sleeve is optionally configured to partially surround the body, e.g. as depicted in FIG. 19A and FIG. 19B. Optionally, the sleeve is configured to partially surround the body and comprises a plurality of prongs (e.g. prongs 35,36 of sleeve 34 depicted in FIG. 19A and FIG. 19B). Optionally, the sleeve comprises a plurality of prongs configured to partially surround the body, wherein the inter-prong spacing is no more than 150°, for example, no more than 120° or no more than 90° (e.g. as depicted by angle phi φ in FIG. 19A,B). Such a sleeve having multiple contact points and/or tailored inter-prong spacing can be contacted by different portions of a user's hand such as different fingers at multiple locations around the sleeve. Such contact by the users hand can be deliberate contact, e.g. in which the sleeve provides a surface for stabilization or movement by the user. Alternatively, such contact can be inadvertent contact. In either scenario, the sleeve contact points or tailored inter-prong spacing can optionally provide a protective barrier to the guide body to prevent the user's hand from contacting the body during use, which could otherwise impede smooth longitudinal movement of the mandrel relative to the guide, e.g. smooth movement necessary to perform certain delicate procedures such, for example, discharge of an implant into the eye or retina. Accordingly, such a configuration surprisingly increases safety, reduces user-error, and increases precision. Such superior advantages can optionally be further enhanced, e.g. when combined with other optional features of the invention such as finger tabs extending from the guide in opposing directions, a butt end configured for abutting against a user's palm, and/or a finger grip provided on the sleeve.

### Finger Grip

Optionally, the sleeve comprises a finger grip disposed about a lateral surface of the sleeve. Optionally, the finger grip comprises lip, a cavity, a friction pad, or a combination thereof. Optionally, the finger grip comprises a thumb grip.

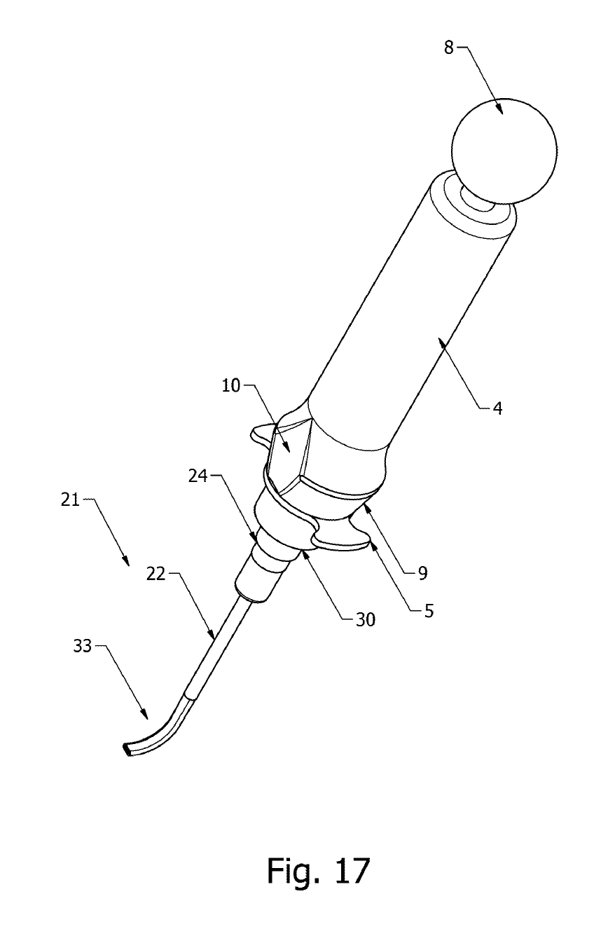

Optionally, the sleeve comprises both a lip and a cavity, wherein the cavity is in the lip, e.g. as depicted in FIG. 17.

Optionally, the lip is a tapered lip (e.g. as depicted in FIG. 17). Optionally, the lip comprises proximal taper (i.e. tapers proximally from the lip peak), a distal taper (i.e. tapers distally from the lip peak), or both.

Optionally, the lip and/or the cavity are configured for interfacing a finger of a hand of the user, e.g. a thumb.

Optionally, the finger grip (e.g. lip and/or the cavity) is configured for interfacing a thumb of the hand of a user (e.g. when the palm of the user's hand interfaces an optional butt end of the discharge unit and/or when at least one finger other than the thumb interfaces at least one optional finger tab of the guide, e.g. as depicted in FIG. 17.

### Butt End

Optionally, the discharge unit comprises a proximal surface configured for interfacing (e.g. abutting against) the palm of a user's hand (‘butt end’).

Optionally, the butt end comprises a convex surface.

Optionally, the butt end is disposed about the proximal end of the discharge unit. For example, the e.g. on the proximal end of the mandrel or the sleeve).

Optionally, the butt end is configured for abutting against the palm of the user. Optionally, abutting the butt end against the palm comprises contacting the butt end with the palm and orienting the device (e.g. a longitudinal axis of the device) substantially perpendicular to the palm. Such an abutted orientation is surprisingly useful in certain procedures, e.g. in contrast to a cylindrical grip or power grip used in prior art devices such as that described in U.S. Pat. No. 4,994,028 to Leonard et al.

The butt end can be configured for abutting against the palm of a user's hand at any location on the palm. Optionally the butt end is configured for abutting against the palm at the distal palmar crease, the proximal palmar crease, or the thenar crease. Optionally the butt end is configured for interfacing the palm at the metacarpus, any of the first, second, third fourth, and fifth metacarpals, or at the base, shaft, or head of any of: the metacarpus, the first, second, third fourth, and fifth metacarpals. Optionally the butt end is configured for interfacing the palm at any of the pretendinous bands. Optionally the butt end is configured for interfacing the palm at the adductor pollicis, the abductor pollicis brevis, or the flexor pollicis brevis. Optionally the butt end is configured for interfacing the palm at the webbing between the thumb and forefinger.

Optionally, the device comprises at least one finger tab extending laterally from the guide, e.g. extending laterally from the guide in opposing directions, and the butt end is configured for abutting against the palm of a user's hand. Optionally, the at least one finger tab comprises a distal surface configured for interfacing at least one finger of the user, e.g. interfacing two fingers situated about opposing surfaces of the guide, when the butt end is abutted against the palm of a user. Optionally, when the device is in the loaded state, the at least one finger tab is offset from the butt end such that the butt end can be abutted against the palm of a user's hand while the user's at least one finger interfaces with the distal surface of the at least one finger tab. Surprisingly, such a device can be configured to provide the user with at least two modes of implant discharge—an injection mode and an ejection mode, wherein the injection mode comprises stabilizing the at least one finger tab and advancing the discharge unit (e.g. by moving a butt end of the discharge unit), and wherein the ejection mode comprises stabilizing the discharge unit by abutting the butt against the palm and retracting the guide by moving the at least one finger tab (e.g. pulling the at least one finger tab with the finger).

Optionally, the device comprise a butt end having a proximal surface and at least one finger tab (e.g. at least one finger tab extending laterally from the guide in opposing directions). Optionally, the distance between the distal surface and the at least one finger tab is configured such that the proximal surface can abutting against the palm of a user's hand while the at least one finger tab is interfaced by at least one finger of the user's hand when the device is in the loaded state and/or the discharged state. Optionally, when device is in the loaded state or when the mandrel is fully retracted, the distance between the at least one finger tab and the proximal surface (‘grip length’) is less than about 13 cm (e.g. less than about 10 cm, less than about 9 cm, or less than about 8 cm). Optionally, the grip length is about 4 cm to about 13 cm, e.g. about 4 cm to about 10 cm, about 4 cm to about 9, about 4 cm to about 8, about 5 cm to about 10 cm, about 5 cm to about 9 cm, about 5 cm to about 8 cm, about 5.5 cm to about 8.5 cm, about 6 cm to about 9 cm, about 6.5 cm to about 8.5 cm, or about 7 cm to about 8 cm. For example, the grip length can be about 7.5 cm

### Movement Limiters

A delivery device of the invention optionally comprises one or more movement limiters. A movement limiter is any mechanism that limits or prevents movement in one or more directions.

Optionally, the device comprises a rotation limiter.

Optionally, the device comprises a retraction limiter.

Optionally, the device comprises a rotation limiter and a retraction limiter.

### Rotation Limiter

Optionally, the delivery device comprises a mechanism for limiting rotation of the mandrel relative to the guide (‘rotation limiter’). The rotation limiter can be any mechanism that prevents or limits rotation of the mandrel relative to the guide. Optionally, the rotation limiter prevents rotation of the mandrel relative to the guide and allows longitudinal movement of the mandrel relative to the guide.

Optionally, rotation limiter is provided by a first member comprised by the guide and a second member comprised by the discharge unit, wherein the first member and second member are disposed circumferentially of each other and configured to move longitudinally relative to each other. Optionally, the first member is a pin comprised by the guide (e.g. extending laterally therefrom) and the second member is a pin track comprised by the sleeve about which the pin moves longitudinally, optionally wherein the pin is recessed in the sleeve (i.e. does not protrude laterally from the sleeve). Optionally, the pin track is a slot or a groove in the sleeve.

A rotation limiter is useful, e.g. in a delivery device comprising and comprises a wide nozzle and/or a mandrel with a wide tip where rotation of the mandrel in the nozzle could damage the mandrel.

### Retraction Limiter

Optionally, the device further comprises a mechanism for limiting retraction of the mandrel relative to the guide (‘retraction limiter’). The retraction limiter can be any mechanism that prevents or limits rotation of the mandrel relative to the guide.

Optionally, the retraction limiter allows retraction of the mandrel relative to the guide up to a distance that provides the device in the loaded state and prevents further retraction. Optionally, the loaded state is the relative position of the mandrel and the guide that retains the implant in the nozzle, substantially flush with the open end of the nozzle.

Optionally, the retraction limiter allows retraction of the mandrel relative to the guide up to a distance that provides the device is in the loaded state and limits retraction to less than an amount that would release contact between the sleeve and the guide body, between the mandrel and the guide, or between an optional plunger head and the guide body.

Optionally, the delivery device comprises a wide nozzle, a wide tip mandrel, and a guide portion that is proximal to the wide nozzle having a diameter substantially less than the tip of the wide tip mandrel and the open end of the wide nozzle, and the retraction limiter allows retraction of the mandrel relative to the guide less than a distance that retracts the wide tip mandrel from the wide nozzle and into the guide portion that is proximal to the wide nozzle having a diameter substantially less than the tip of the wide tip mandrel.

### Optional Configurations and Dimensions

Devices of the present invention may be customized in different sizes and shapes, e.g. for different implants, subjects, users, and methods of manipulating the device. Additionally, device components, e.g. mandrels and guides, can be customized in different sizes and shapes delivery of different kinds of implants such as tissue, gels containing different trophic factors or drugs, electronic microchips, and other implantable substances.

The mandrel and guide may be constructed of any length, as long as they together form a functional delivery device. Optionally, the guide has a longitudinal length of about 5 cm to about 14 cm. Optionally, the length of sleeve that makes contact with the guide is about 2 cm to about 9 cm. Optionally, at least a portion of the guide (e.g. an insertable length) comprises an outer diameter of less than about 0.4 cm and a longitudinal length of about 1 cm to about 5 cm (e.g. about 1.5 cm to about 3.5 cm) or about 0.5 cm to about 2 cm.

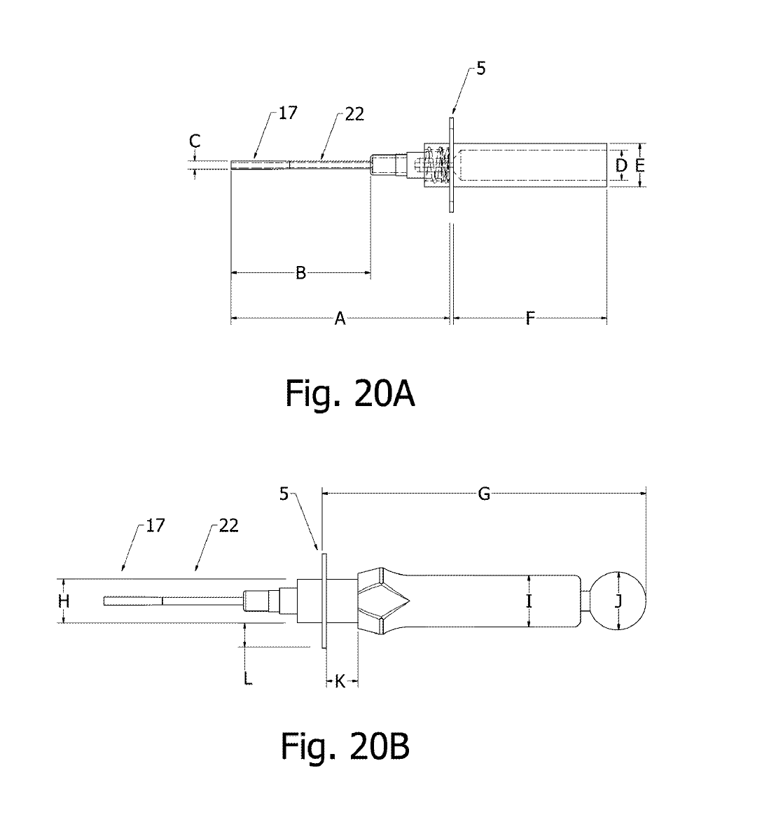

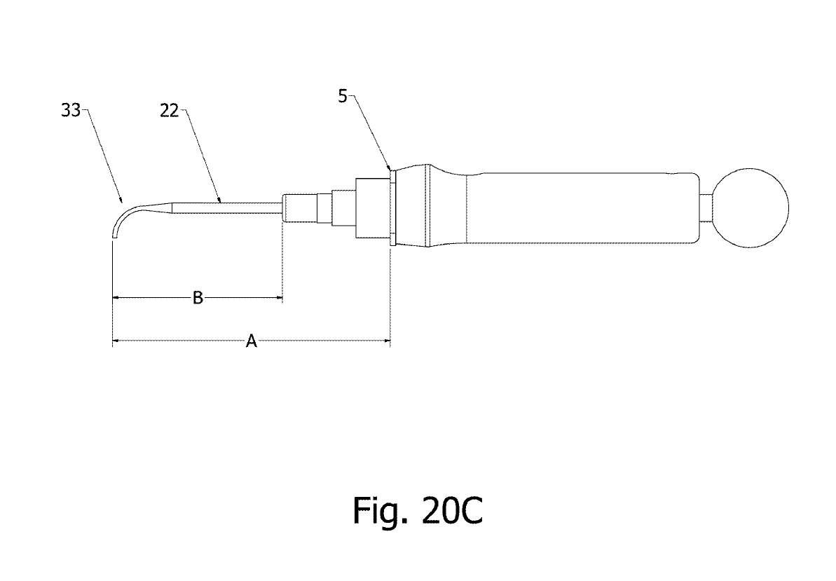

As depicted in FIG. 20A, FIG. 20B, and FIG. 20C, a device of the present invention optionally comprises one or more dimensions A-K listed in Table 2. Optionally, the device comprises one or more or all dimensions selected from Dimensions A-L of Example X. Optionally, the device comprises one or more or all dimensions selected from Dimensions A-L of Example Y. Optionally, the device comprises one or more or all dimensions selected from Dimensions A-L of Example Z, or dimensions within 20% (i.e. +/−20%) thereof.

TABLE 2
Device Dimensions

| Dimension Label | Dimension Description | Example X | Example Y | Example Z | |
|---|---|---|---|---|---|
| A | length of finger tab to distal end of nozzle | ≦10 cm | ≦7 cm | 4.5 cm, 5.1 cm, or 6.4 cm | |
| B | insertable length | ≧1.7 cm | ≧1.7 cm | 1.9 cm, 2.6 cm, or 3.8 cm | |
| C | insertable diameter | ≦3 cm | ≦2.5 | 1.4 | mm |
| D | body inner diameter or plunger diameter | 3 mm-15 mm | 3 mm-15 mm | 7 | mm |
| E | body outer diameter or sleeve inner diameter | 0.3 cm-5 cm | 0.5 cm-2 cm | 0.95 | cm |
| F | length of body that makes sliding contact with sleeve | 2 cm-9 cm | 3 cm-6 cm | 4.3 | cm |
| G | length from butt end to finger tab | ≦10 cm | 4 mm-9 cm | 6.8 | cm |
| H | outer guide diameter distal of finger tabs | ≦5 cm | ≦3.5 cm | 0.95 | cm |
| I | outer diameter of sleeve | 0.3 cm-5 cm | 0.5 cm-3 cm | 1.13 | cm |
| J | diameter of butt end | 0.5 cm-4 cm | 0.5 cm-2 cm | 1.27 | cm |
| K | maximum retraction length | ≦3 cm | ≦3 cm | 0.7 | cm |
| L | finger tab lateral extension | ≧0.3 cm | 0.3-2.5 cm | 0.65 | cm |

Optionally, a device of the invention comprises a tailored length of the finger tab to the distal end of the nozzle (Dimension A in Table 2). Optionally, the length is tailored such that the user, while placing his finger(s) on the finger tab(s), can stabilize his hand about a subjects surface (e.g. head) while the distal end of the nozzle is located at the target site (e.g. retina). Optionally, the length of the dimension A is no greater than about 11 cm, no greater than about 10 cm, no greater than about 8 cm, no greater than about 7 cm, or no greater than about 6 cm. Optionally, the length of dimension A is no greater than about than about 11 cm or no greater than about 8 cm, and the device is used to deliver an implant to the retina of a mammal such as an ungulate (e.g. horse), a primate (e.g. human), or a rodent (e.g. rat). Optionally, the length of dimension A is no greater than about than about 10 cm, no greater than about 7 cm, or no greater than about 6 cm, and the device is used to deliver an implant to the retina of a mammal (e.g. human). Optionally, the length of dimension A is no greater than about than about 10 cm, no greater than about 7 cm, or no greater than about 6 cm, and the device is used to deliver an implant to the retina of a mammal (e.g. human or companion animal such as a dog). Optionally, the length of dimension A is no greater than about than about 9 cm, no greater than about 6 cm, or no greater than about 5 cm, and the device is used to deliver an implant to the retina of a rodent (e.g. rat).

Optionally, a device of the invention comprises a tailored insertable length (Dimension B in Table 2). The insertable length is a portion of the guide which is configured for insertion in a subject, e.g. without causing undue injury to the subject, such as a portion of the guide that does not comprise abrupt changes in diameter. Optionally, the insertable length is tailored such that the distal end of the nozzle can reach the target site (e.g. the retina) when the nozzle is inserted through a surface of the subject (e.g. the eye surface). Optionally, the insertable length is at least about 0.35 cm, at least about 1.7 cm, at least about 2.5 cm, or at least about 3.5 cm. Optionally, the insertable length is at least about 1.7 cm and the device is used for delivering an implant to the retina of a mammal such as a human (e.g. adult human or immature human such as an infant or other human without full grown eyes), an ungulate (e.g. a horse), a companion animal (e.g. a dog), or a rodent (e.g. a rat). Optionally, the insertable length is at least about 2.5 cm and the device is used for delivering an implant to the retina of a human (e.g. adult human), a companion animal (e.g. a dog), or an ungulate (e.g. a horse). Optionally, the insertable length is at least about 3.5 cm and the device is used for delivering an implant to the retina of an ungulate (e.g. a horse).

Optionally, a device of the invention comprises a tailored insertable diameter (Dimension C in Table 2). The insertable diameter is the greatest diameter of the portion of the guide configured for insertion. Optionally, the insertable diameter is tailored for insertion through the surface of an eye. Optionally, the insertable diameter is no greater than about 3 mm, no greater than 2.5 mm, no greater than 2 mm, no greater than 1 mm, or no greater than 0.8 mm. Optionally, the insertable diameter is no greater than about 3 mm or no greater than 2.5 mm and the device is used for delivering an implant into the eye of a mammal such as a human, an ungulate (e.g. a horse), a companion animal (e.g. a dog), or a rodent (e.g. a rat). Optionally, the insertable diameter is no greater than about 2.5 mm or no greater than 2 mm and the device is used for delivering an implant into the eye of a mammal such as a human, a companion animal (e.g. a dog), or a rodent (e.g. a rat). Optionally, the insertable diameter is no greater than about 1 mm or no greater than 0.8 mm and the device is used for delivering an implant into the eye of a rodent (e.g. a rat or a mouse).

Optionally, a device of the invention comprises a tailored body inner diameter or plunger diameter (Dimension D in Table 2). Optionally, the body inner diameter or plunger is tailored to provide a rigid or semi-rigid member to impart force on a push rod. Optionally, dimension D is about 3 mm to about 15 mm.

Optionally, a device of the invention comprises a tailored body outer diameter or sleeve inner diameter (Dimension E in Table 2). Optionally, the body outer diameter and sleeve inner diameter is tailored to such that it is substantially greater (e.g. at least 2 time greater or at least 5 times greater) than the outer diameter of the nozzle to provide a sliding contact surface area between the sleeve and the body that allows smooth and guided longitudinal movement of the discharge unit relative to the guide. Optionally, the body outer diameter and sleeve inner diameter is about 0.3 cm to about 5 cm, e.g. about 0.5 cm to about 3 cm, or about 0.5 cm to about 2 cm.

Optionally, a device of the invention comprises a tailored length of body that makes sliding contact with sleeve (Dimension F in Table 2). Optionally, the length of the body that makes sliding contact with sleeve is substantially greater (e.g. at least five times greater) than the maximum retraction distance (Dimension K). Such a configuration provides, for example, smooth and guided longitudinal movement of the discharge unit relative to the guide and, for example, allowing other optional technical features of the invention such as the ability to abut the a proximal surface of the device against a user's palm. Optionally, the length of the body that makes sliding contact with sleeve is about 2 cm to about 9 cm (e.g. about 3 cm to about 6 cm).

Optionally, a device of the invention comprises a tailored length from the butt end to finger tab when the device is in the loaded position (Dimension G in Table 2). Optionally, Dimension G is tailored such that the user can place one or more fingers on the finger tab and abut the butt end against is palm. Optionally, Dimension G is less than about 13 cm (e.g. less than about 10 cm, less than about 9 cm, or less than about 8 cm). Optionally, Dimension G is about 4 cm to about 13 cm, e.g. about 4 cm to about 10 cm, about 4 cm to about 9, about 4 cm to about 8, about 5 cm to about 10 cm, about 5 cm to about 9 cm, about 5 cm to about 8 cm, about 5.5 cm to about 8.5 cm, about 6 cm to about 9 cm, about 6.5 cm to about 8.5 cm, about 6 cm to about 8 cm, or about 7 cm to about 8 cm. For example, Dimension G is optionally about 7.5 cm.

Optionally, a device of the invention comprises a guide having a tailored outer diameter distal of finger tabs (Dimension H in Table 2). Optionally, Dimension H is configured such that the user can split his fingers around the guide distal of the finger tabs and place the fingers on distal surface of finger tabs extending in opposing directions from the guide. Optionally, Dimension H is no greater than about 5 cm, e.g. no greater than about 4 cm, no greater than about 3 cm, no greater than about 2 cm, or no greater than 1.5 cm.

Optionally, a device of the invention comprises a tailored outer diameter of sleeve (Dimension I in Table 2). Optionally, the outer diameter of the sleeve is configured such that the device can comfortably and precisely be handled in one or ways taught herein. Optionally, Dimension I is about 0.3 cm to about 5 cm, e.g. about 0.5 cm to about 3 cm, or about 0.5 cm to about 2 cm.

Optionally, a device of the invention comprises a tailored butt end diameter (Dimension J in Table 2). Optionally, the butt end diameter is tailored such that it can be abutted and secured against the palm of a user's hand. Optionally, Dimension J is about 0.5 cm to about 5 cm, e.g. about 0.5 cm to about 3 cm, about 0.5 cm to about 2 cm, or about 1 cm to about 2 cm.

Optionally, a device of the invention comprises a tailored maximum retraction length (Dimension K in Table 2). Optionally, the maximum retraction length (e.g. set by a retraction limiter) is tailored for a desired implant, e.g. an ocular implant. Optionally, Dimension K is no greater than about 3 cm, no greater than about 2 cm, or no greater than about 1 cm.

Optionally, a device of the invention comprises at least one finger tab having a tailored lateral extension (Dimension L in Table 2). For example, the finger tab lateral extension can be configured to provide a distal surface for interfacing one or more fingers. Optionally, the finger tab lateral extension is at least 0.3 cm. Optionally, the finger tab lateral extension is about 0.3 cm to about 2.5 cm.

Optionally, a device of the invention comprises a Dimension A no greater than about than about 10 cm and a dimension B of at least about 3.5 cm. Such a device is optionally used for delivering an implant to the retina of a horse. Optionally, a device of the invention comprises a Dimension A no greater than about 7 cm and a dimension B of at least about 2.5 cm. Such a device is optionally used for delivering an implant to the retina of a human (e.g. adult human). Optionally, a device of the invention comprises a Dimension A no greater than about 6 cm and a dimension B of at least about 1.7 cm. Such a device is optionally used for delivering an implant to the retina of a human (e.g. an immature human). Optionally, a device of the invention comprises a Dimension A no greater than about 6 cm and a dimension B of at least about 0.35 cm. Such a device is optionally used for delivering an implant to the retina of a rodent (e.g. rat or mouse).

Devices of the present invention are not limited to any particular size. For example, a device taught above, or any component thereof such as the mandrel, guide, and nozzle, or any dimension thereof, such as lateral dimension or longitudinal dimension may be scaled down to 1%-10%, such as 1% or 10% (e.g. for prenatal applications), or may be scaled up to 500%-1500% (for applications in large animals such as elephants). Also envisioned is a device scaled up or down (e.g. 1%-10% or 500%-1500) in the lateral dimensions (e.g. to facilitate more or less bulky implants or for insertion into smaller or larger environments) but not substantially scaled in the longitudinal dimension. Also envisioned is a device scaled down (e.g. 1%-10% or 30%-60%) in the lateral dimensions (e.g. to facilitate entry into a vein or artery) but scaled up (e.g. 500%-1500%) in the longitudinal dimension (e.g. of the nozzle) to deliver an implant to target site that is remote from the insertion site.

The devices taught herein are not limited to any particular configuration or device components and modifications which alternatively provide components having the same property or function are also contemplated. Accordingly, the invention provides an alternative embodiment in which a component that performs a specific function is replaced by a means for performing said function.

### Discharge Modes

A delivery device of the present invention can be configured for any mode of discharging an implant. Optionally, the device is configured for injection, ejection, or both injection and ejection.

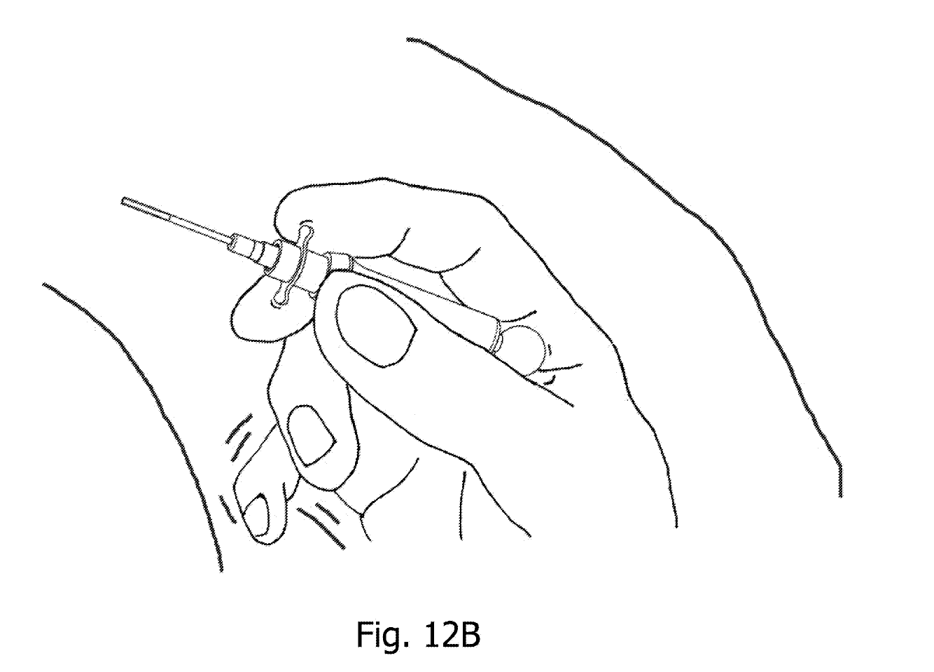

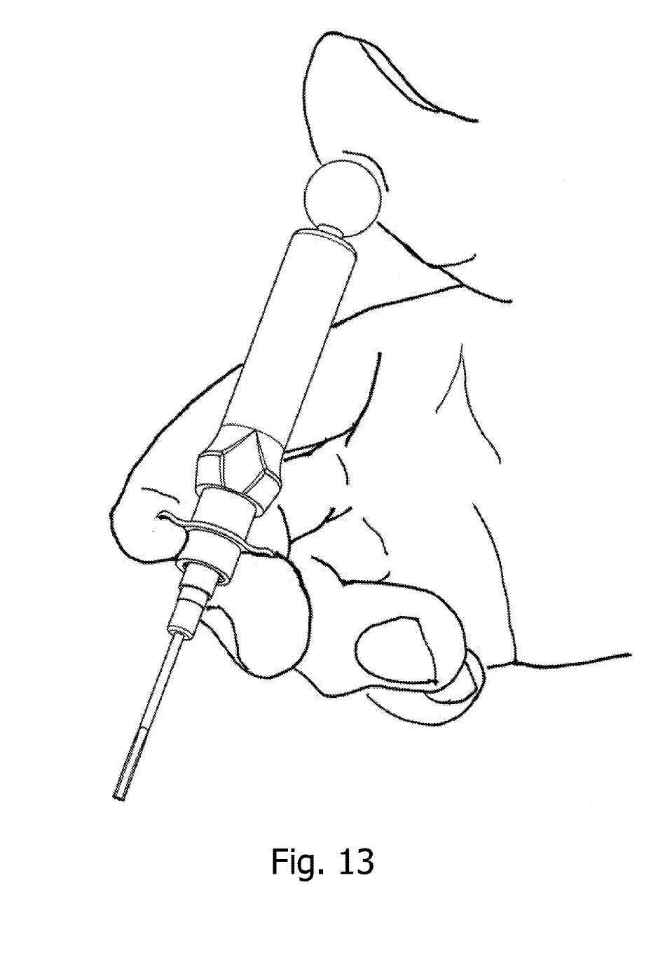

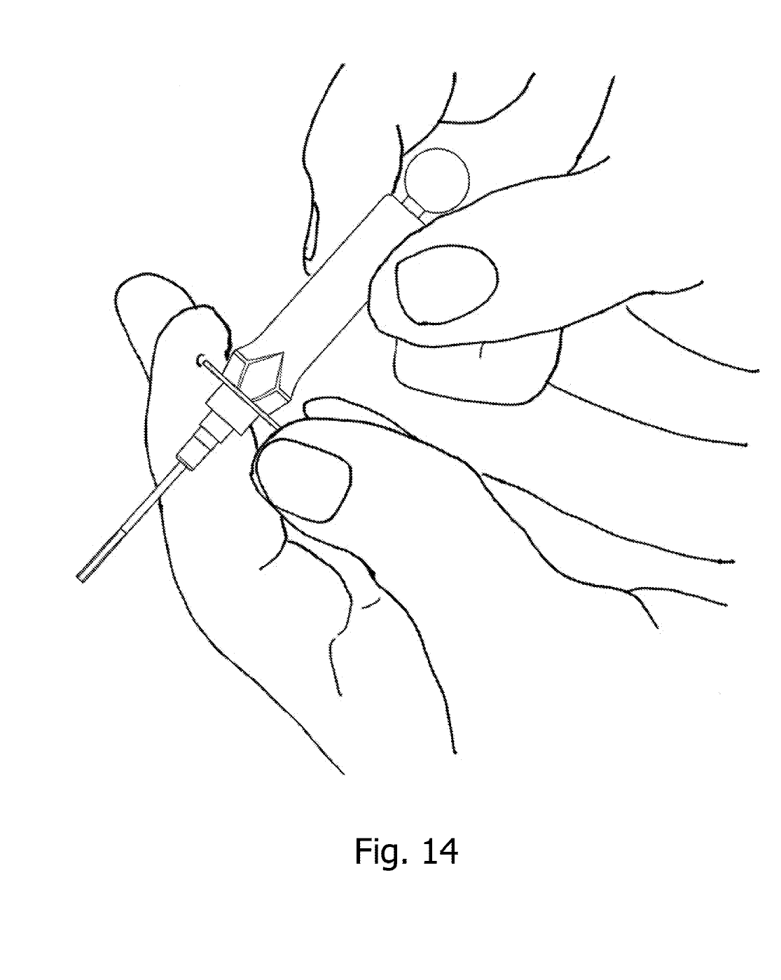

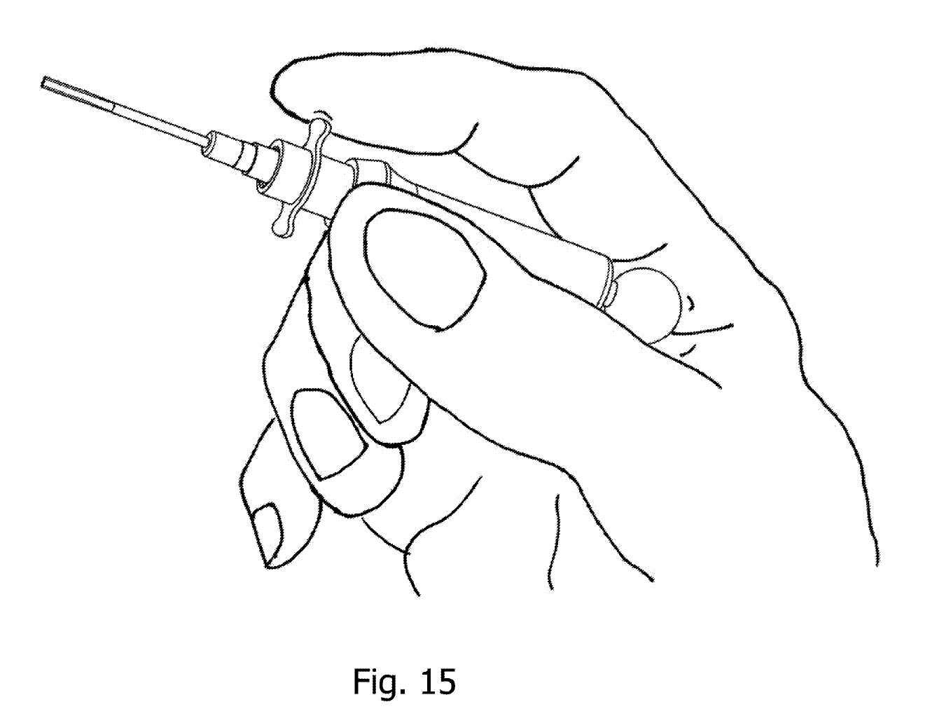

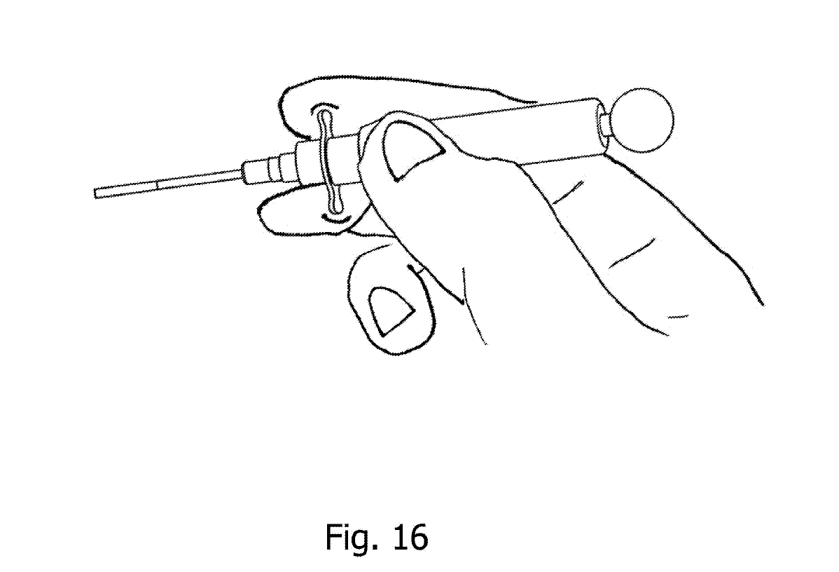

Optionally, the device is configured for injection. Injection comprises stabilizing the guide and advancing the mandrel (e.g. via the butt end) to discharge the implant from the open end of the guide. Examples of injection modes of discharge are illustrated in FIG. 12 and FIG. 13, in which a surface (e.g. the subject) is used to stabilize a user's fingers or hand, which in turn stabilize the finger tabs of the guide.

Optionally, the device is configured for ejection. Ejection comprises stabilizing the mandrel and retracting the guide (e.g. via at least one finger tab) to discharge the implant from the open end of the guide. Examples of ejection modes are illustrated in FIG. 11 and FIG. 15 in which the butt end of the discharge unit is stabilized against the user's palm. As depicted in FIG. 11 and FIG. 15, optionally, the device is configured such that the user can additionally or alternatively stabilize the guide using a thumb (e.g. against a finger grip such as a lip and/or cavity).

Optionally, device is configured for providing the user with at least two modes of implant discharge—an injection mode and an ejection mode (‘dual mode device’), e.g. such that the user can select which mode, injection or ejection, to deliver an implant. Examples of such a device are detailed in Example 1 and Example 2. As illustrated in FIG. 11 and FIG. 12 the same device can be used in ejection mode or injection mode, respectively. Surprisingly, certain embodiments taught herein allow the user to choose either injection or ejection, or even a combination of the two (e.g. partial injection followed by ejection) to discharge an implant. As an example, a physician using an injection mode may place the open distal end of the nozzle at the periphery of the target site and inject or “push” the implant into the target site. In contrast, an ejection mode may comprise inserting the nozzle into the target site such that the implant is already in the desired location before “backing out” the nozzle and leaving the implant at the target site. It has been discovered by the inventors that the preference or ability to use injection versus ejection modes can be physician-dependent or procedure dependent. Many factors can govern the choice of injection versus ejection modes including dexterity of the user, orientation of the device to the subject, the target site (e.g. location, environment, or morphology of the target site), or the implant itself (e.g. strength, shape, or size). Accordingly, devices of the present invention are surprisingly useful in a huge array of scenarios that would otherwise require a plurality of devices. The present invention teaches several features that can be used alone or in concert to provide a dual mode device, e.g. a device having 1) at least one finger tab extending from the guide in opposing directions and/or having a distal surface for interfacing a finger, and 2) a sleeve providing multiple contact points and/or a discharge unit having a butt end configured for abutting a palm of the user's hand while finger(s) of the hand interface the at least one finger tab.

### Manufacture

A delivery device of the invention can be made by any method.

Optionally, the delivery device is formed from a syringe barrel, a syringe needle, a syringe plunger, or a combination thereof. Such “syringe” components can optionally be off-the-shelf syringe components that are then used to manufacture a delivery device of the invention.

Optionally, a method of manufacturing a delivery device of the present invention comprises the steps of:
a. providing a syringe barrel;
b. providing a syringe needle;
c. providing a syringe plunger;
d. providing a nozzle;
e. providing a sleeve configured to accept the syringe barrel;
f. providing an elongated member configured for insertion through the syringe needle and the nozzle (‘push rod’);
g. inserting the syringe plunger in the sleeve;
h. attaching the plunger to the sleeve;
i. attaching the push rod to the syringe plunger;
j. wherein the push rod is attached to the syringe plunger arm at a location distal of the attachment point of the sleeve and the plunger;
k. attaching the nozzle to the syringe needle;
l. attaching the syringe needle to the syringe barrel; and
m. inserting the mandrel in the guide.

Optionally, the syringe barrel has finger tabs at the distal end.

Optionally, the syringe barrel comprises a luer lock or a slip tip.

Optionally, the method further comprises removing a proximal portion of the syringe barrel.

Optionally, the method further comprises sterilizing the delivery device. Optionally, sterilizing comprises heat sterilization or heatless sterilization. Examples of useful sterilization methods include steam sterilization, dry heat sterilization, autoclaving, ionizing radiation (e.g. gamma or electron-beam radiation), and gas sterilization (e.g. ethylene oxide or formaldehyde).

Optionally, the method further comprises loading an implant in the guide. Optionally, the implant is an ocular implant.

Optionally, the method further comprises packaging the delivery device.

Optionally, the method further applying a cover over the nozzle (e.g. a rigid cover).

The invention also provides a product or delivery device made by the method of manufacture.

### Products

A delivery device of the present invention can optionally be packaged to provide a packaged product. Accordingly, the invention provides a product comprising a sealed package comprising the device.

Optionally, the product further comprises an implant loaded in the device.

Optionally, the sealed package is sterile.

Optionally, the implant is comprises an ocular plant, retinal cells (e.g. retinal epithelial cells), a biodegradable fibrotic membrane, small intestine submucosa, an SU-8 scaffold, an epoxy resin, an epoxy based viscous polymer, or a micropatterned implant.

Optionally, the implant comprises a plurality of pores. Optionally, the implant comprises a substrate (e.g. sheet or membrane) comprising a plurality of pores. For example, the implant optionally comprises an epoxy-based viscous polymer, such as an SU-8 substrate, comprising a plurality of pores. As a non-limiting example, the implant can comprise 8 micron thick layer of epoxy-based polymer (e.g. SU-8) having pores with a diameter of about 5 microns and further comprising RPE cells.

Optionally, the implant comprises an epoxy resin. Optionally, the epoxy resin is a photoractive epoxy resin. Optionally, the epoxy resin comprises a viscous epoxy resin (e.g. a viscous SU-8 resin). Optionally, the epoxy resin comprises a Bisphenol resin (e.g. Bisphenol A or Bisphenol F resin) such as a diglycidyl ether of a Bisphenol. Optionally, the epoxy resin comprises an EPON resin such as SU-8, which comprises a glycidyl ether of bisphenol-A novolac.

Optionally, the implant comprises a photoreactive resin. Optionally, the photoactive resin is a photoactive epoxy resin, e.g. an SU-8 photoactive epoxy resin. Optionally, the implant comprises an epoxy resin (e.g. an EPON epoxy resin such as SU-8), a solvent, and a photoinitiator. Optionally, the photoactive resin comprises a multifunctional epoxy resin, a multifunctional acrylate resin, or a resin having two or more functional groups and containing an oxirane group. Optionally, the photoactive resin comprises a bisphenol A epoxy resin, a bisphenol F epoxy resin, a hydroquinone epoxy resin, a resorcinol epoxy resins, or a novolac epoxy resin.

### Kits

A delivery device of the present invention can be provided with a plurality of alternate nozzles. Accordingly, the invention provides a kit comprising a device or a product of the invention, and a plurality of alternate nozzles. With a kit of the invention, the user can optionally select a nozzle of the plurality of nozzles and couple the nozzle to the body to form an assembled guide.

Optionally, the plurality of alternate nozzles comprises nozzles having different shapes or sizes. Optionally, the plurality of alternate nozzles comprises nozzles configured for different procedures, different implants, different subjects, or different target sites.

Optionally, the plurality of alternate nozzles comprises nozzles having different implants loaded therein.

### Methods

A delivery device of the present invention can be used in any method. One method of the invention comprises using the delivery device delivering an implant to a target site in a subject (e.g. patient).

Optionally, the method comprises injecting or ejecting the implant from the guide. Optionally, the method comprises selecting a mode of discharge from injection and ejection.

Optionally, the method comprises delivering an implant (e.g. cells) to the subretinal space of a patient's eye.

Optionally, the method comprises steps of loading the implant (e.g. cells) into the guide, inserting the guide through the surface of the eye, orienting the open end of the guide in proximity to the retina, sliding the open end of the guide under the retina and into the subretinal space, and discharging the implant by advancing the mandrel relative to the guide.

Devices of the present invention are optionally useful for delivering implants to a target site in a subject. Devices of the present invention are optionally further useful for delivering implants target site is blocked or partially blocked by a delicate environment and/or the is fragile.

Optionally, the subject is an organism. Optionally, the organism is an animal, optionally a mammal, optionally a human or horse. Optionally, the mammal is a companion mammal, for example, a cat or dog. Optionally, the mammal is a laboratory mammal, for example, a mouse or a rat. Optionally, the target site is an organ, cavity, or joint. Optionally, the target is an eye (e.g. the subretinal space of the eye).

Optionally, the implant is a tissue specimen, cellular specimen, an electronic chip, a drug, or a gel. Optionally, the tissue specimen is nervous, cardiac, vascular, bone, joint tissue, or the like, for example, radioactive seeding. Optionally, the nervous tissue is ocular tissue, for example, retinal tissue (e.g. fetal retinal tissue).

Optionally, the target site is an electronic device or a mechanical device, such as a circuit board, computer or computer component, automobile, medical implant (e.g. prosthetic tissue or organ).

Some delivery devices of the present invention provide a delivery device having a removable nozzle. Optionally, the nozzle is substantially non-cylindrical (e.g. a wide nozzle) and guide segment proximal of the nozzle is cylindrical. Optionally, the target site is an eye (e.g. subretinal space).

Some delivery/extraction devices of the present invention provide a device having a removable or disposable nozzle. Accordingly, one embodiment of the present invention provides a method of delivering an implant, the method comprising assembling the guide by coupling a nozzle to a guide body, placing the open end of the guide at the target site, and discharging the implant. Optionally, the implant comprises ocular tissue, such as retinal tissue (e.g. RPE cells). Optionally, the implant comprises RPE cells. RPE cells can be made, e.g. by spontaneous differentiation or by a method taught by Singh et al. (“Functional Analysis of Serially Expanded Human iPS Cell-Derived RPE Cultures” IOVS, October 2013, Vol. 54, No. 10, pp 6767-6778)

Certain embodiments of the present invention are especially useful for ocular surgery because target site is blocked or partially blocked by a delicate environment and the implant intended for extraction or delivery is often fragile. Accordingly, one embodiment of the present invention provides a method for delivering an implant from a device of the present invention to a target site in an ocular environment (e.g. subretinal space), the method comprising loading an implant into the guide, inserting the guide into an eye, orienting the open end of the guide at a target site in the eye, and advancing the mandrel relative to the guide to discharge the implant at the target site. Optionally, the target site is the subretinal space of the eye. Optionally, the method comprises making an incision in the eye before inserting the mandrel guide through the incision. Optionally, the method comprises inserting the mandrel guide through the pars plana in the region of the ciliary body at the periphery of the retina. Optionally, the method comprises incising the retina near the diseased target site giving access to the subretinal space. Optionally, advancing the mandrel relative to the guide comprises injection or ejection of an implant. Optionally, the device is a device as detailed in Example 1 or Example 2.

Optionally, the method comprises delivering an implant to a human eye and the nozzle is curved.

Optionally, the method comprises delivering an implant to a non-human animal (e.g. a rodent such as a mouse or rat) and the nozzle is straight.

Optionally, the method comprises inserting the nozzle of the device into the eye and delivering an implant to the eye (e.g. the retina or subretinal space). Optionally, the nozzle is a wide nozzle (e.g. ultra wide nozzle) and the step of inserting the nozzle in to the eye comprises inserting the nozzle through the sclera. Optionally, the nozzle is a cylindrical nozzle or has a circular cross section, and the step of inserting the nozzle in to the eye comprises inserting the nozzle through the cornea.

Optionally, a method of using a device of the invention comprises discharging cells, tissue, or other implant into the subretinal space through the sclera, entering posterior to the equator in order to detach the fovea and inject into the subretinal bleb.

Optionally, a method of using a device of the invention comprises discharging cells, tissue, or other implant under the foveal retina, e.g. in a small bleb, of a subject, through the sclera, e.g. retrobulbarly. Surprisingly, advantages of certain embodiments include the use of a nozzle tip that is sturdy enough to penetrate the sclera and at the same time has the appropriate curve and flexibility to pass under the retina, without the tip damaging the retina, to deliver the implant to the foveal area where a disease process is in need of therapy.

### EXAMPLES

### Example 1

### Delivery Device Comprising a Sleeve

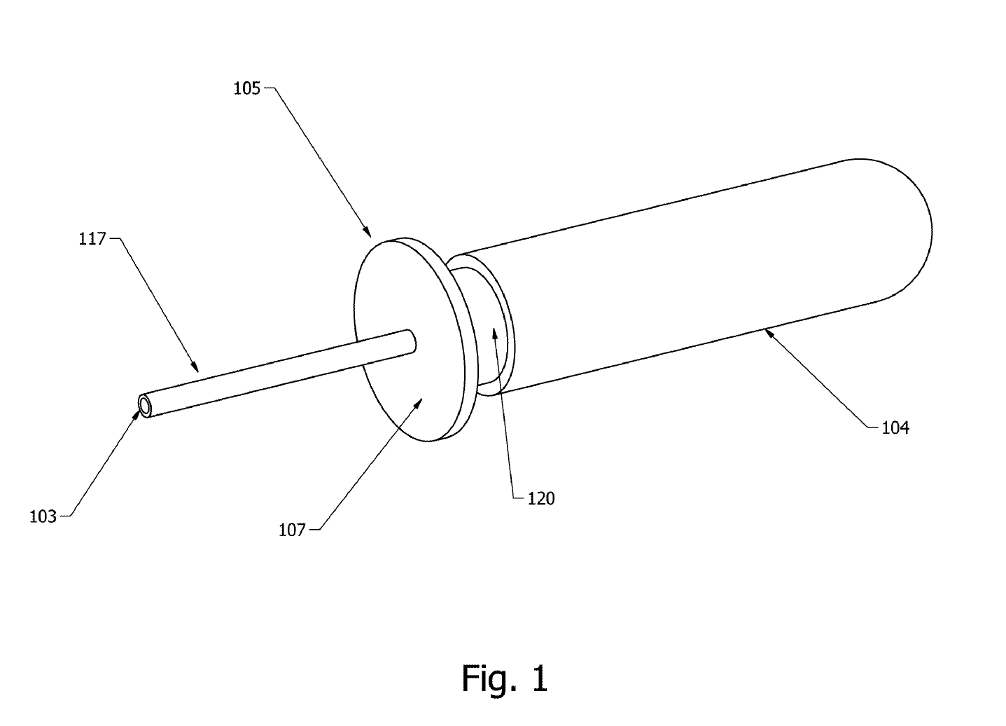

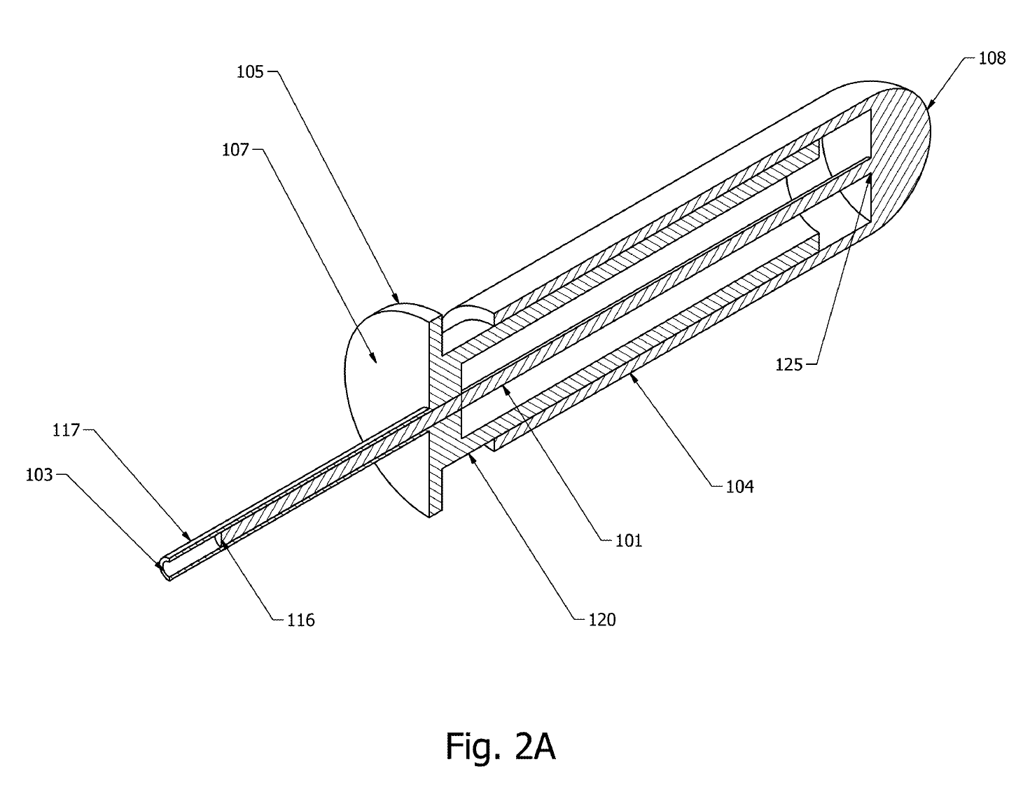

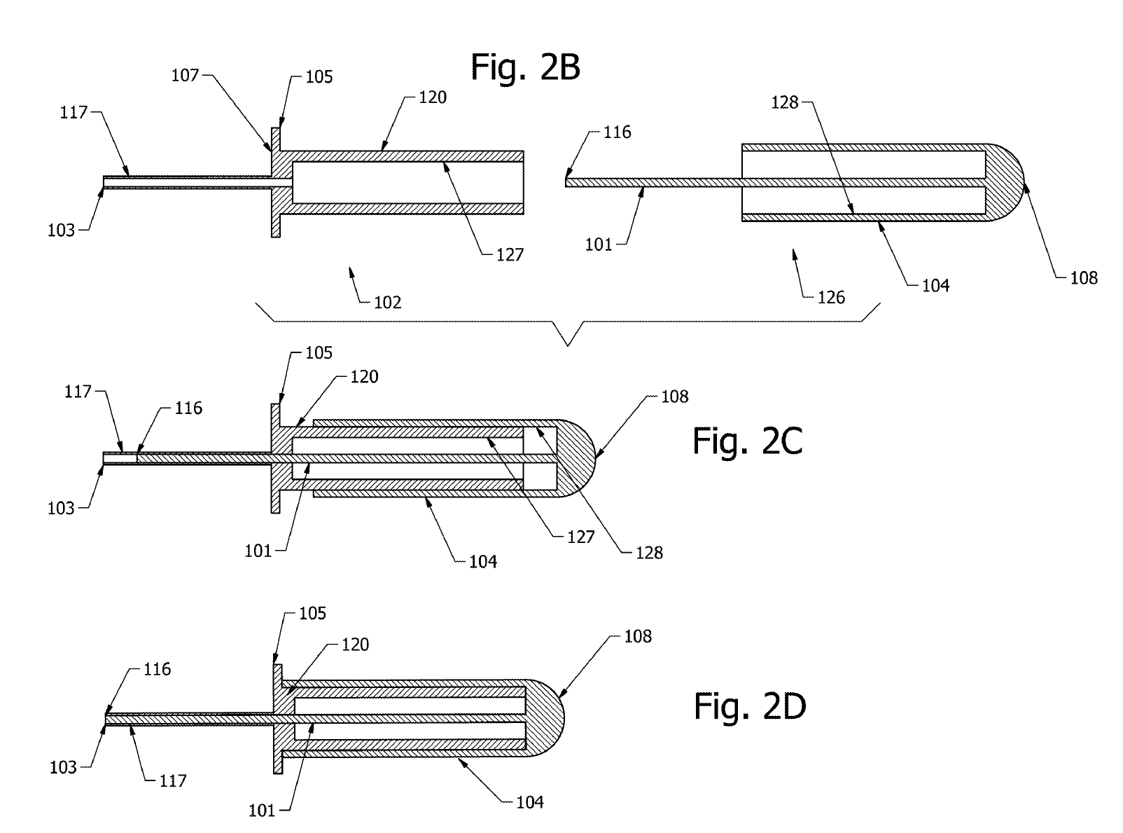

FIG. 1 depicts a delivery device of the invention. FIG. 2A depicts a sectioned perspective view of the device in the loaded state. FIG. 2B, FIG. 2C, and FIG. 2D depict sectioned elevation views of the device in an exploded view, a loaded state, and a discharged state, respectively.

The device comprises a mandrel 101 and a sleeve 104 which together form the discharge unit 126. The device further comprises a nozzle 117 and a body 120 which together form a discharge unit guide (‘guide’) 102. The nozzle 117 comprises an open distal end 103 and can be loaded with an implant (implant not shown). The mandrel 101 is disposed internally to the guide 102 and is configured to move relative to the guide 102 to discharge a loaded implant from the open end 103. The sleeve 104 is disposed externally to the body 120 of the guide 102 and is attached to the proximal end 125 of the mandrel 101 for longitudinal movement therewith relative to the guide 102 (i.e. advancement or retraction relative to the open distal end 103).

The device further comprises a finger tab 105 extending laterally from the body 120 of the guide 102 in opposing directions. Specifically, as depicted, finger tab 105 is configured as a ring tab that surrounds the body 120. The finger tab 105 comprises a distal surface 107 configured for interfacing two fingers situated about opposing surfaces of the guide 102.

The discharge unit 126 comprises a butt end 108 having a convex surface. As depicted, the butt end 108 is disposed at the proximal end of the discharge unit 126.

The device is configured (i.e. sized and shaped) such that the butt end 108 can be abutted against the palm of a user's hand while the user curls his fingers over the finger tab 105 to interface the distal surface 107 of the finger tab 105. In this orientation, discharge of the implant occurs upon the user pulling his fingers towards his palm to retract the guide 102 relative to the mandrel. Accordingly, this device allows the user to discharge the implant via injection or ejection, wherein injection comprises stabilizing the finger tab 105 and advancing the discharge unit 126 (e.g. by applying a force to the butt end 108) and wherein ejection comprises stabilizing the discharge unit 126 (e.g. by interfacing the sleeve 104 or the butt end 108) and retracting the guide 102 (e.g. by applying a force to the finger tab 105).

The luminal (i.e. inner) surface 128 of the sleeve 104 makes sliding contact with the body 120 about a large and elongated surface area, thereby providing smoothly guided longitudinal movement of the discharge unit 126 relative to the guide 102. 00103 The sleeve 104 is configured such that the surface area of sliding contact between the body 120 and the luminal surface 128 of the sleeve 104 increases as the sleeve 104 is advanced relative to the body 120. As can be seen by comparing the longitudinal position of the mandrel distal end 116 relative to the open end of the nozzle 103 in loaded state and the discharged state depicted in FIG. 2C and FIG. 2D, respectively, the longitudinal length of the elongated surface area that makes sliding contact between the sleeve 104 and the body 120 is substantially greater (e.g. at least five times greater) than the distance of longitudinal movement required to discharge a loaded implant. As can be further seen, the contact diameter of the body 120 and sleeve 104 (i.e. outer diameter of the body and inner diameter of the sleeve which form the surface area making sliding contact) is substantially greater (e.g. at least 2 time greater or at least 5 times greater) than the outer diameter of the distal end of the nozzle 117. Independently or combined, the length and diameter of the sliding contact surface area between the sleeve 104 and the body 120 provides an extremely smooth and guided longitudinal movement of the discharge unit 126 relative to the guide 102.

### Example 2

### Delivery Device

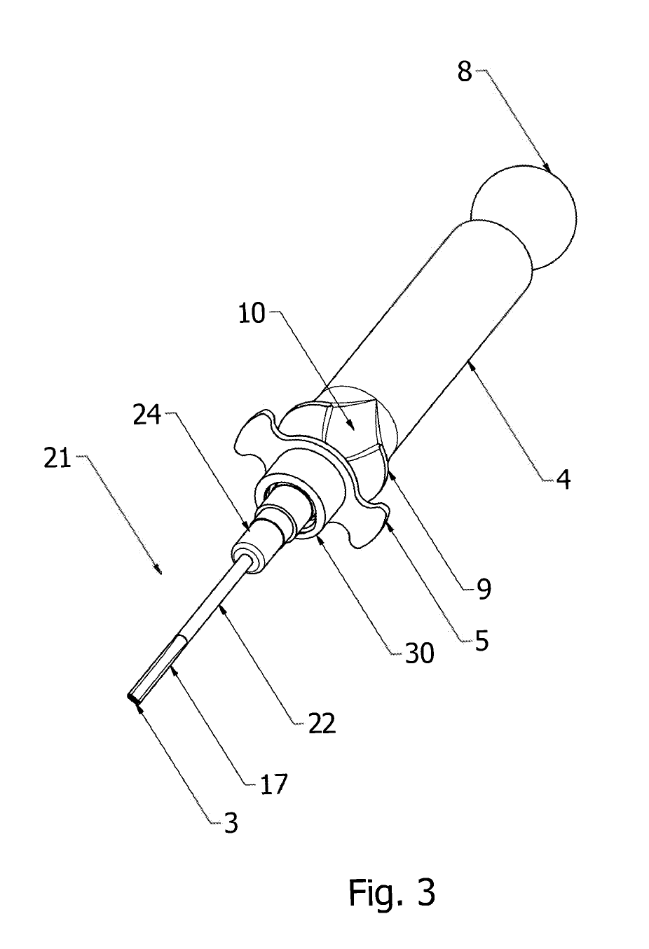

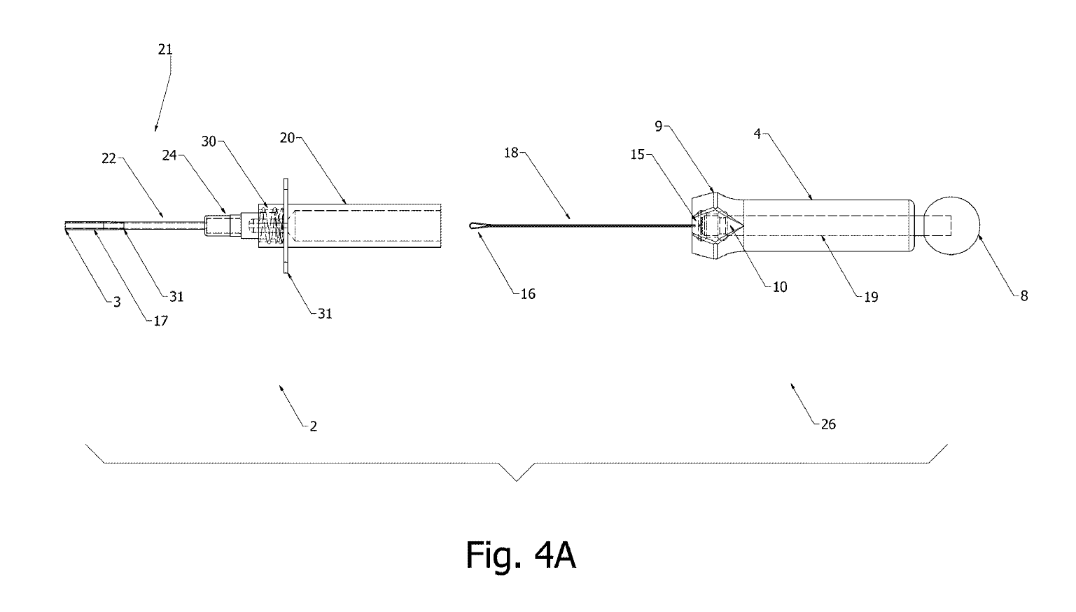

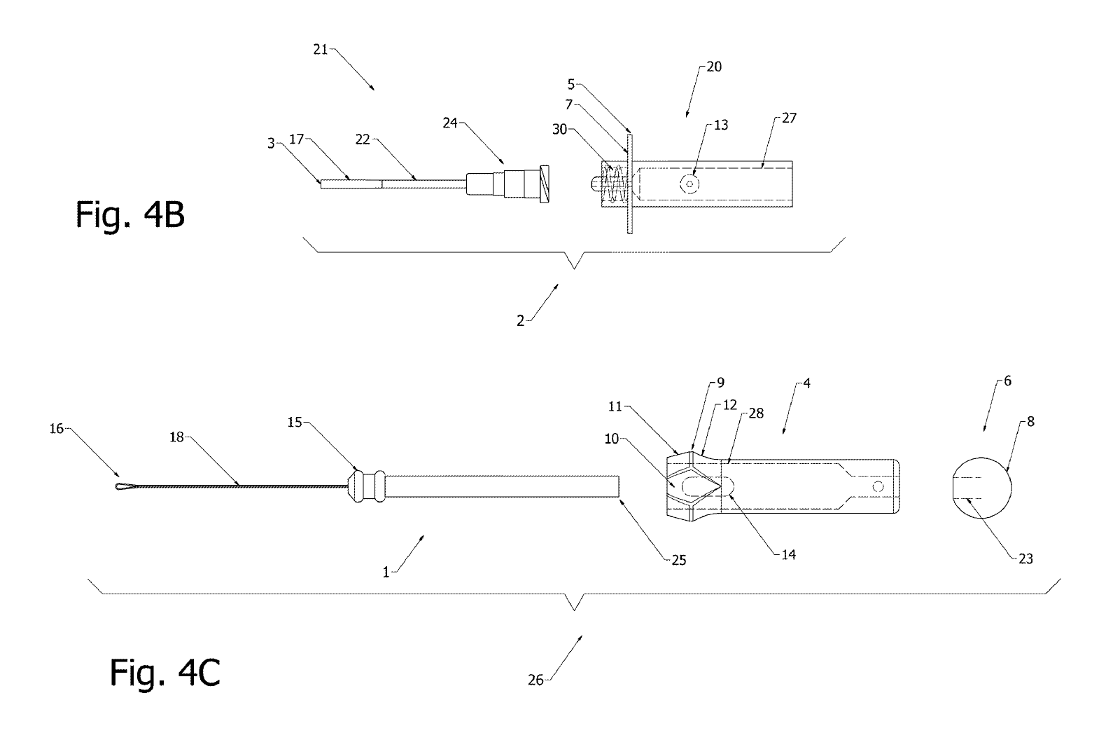

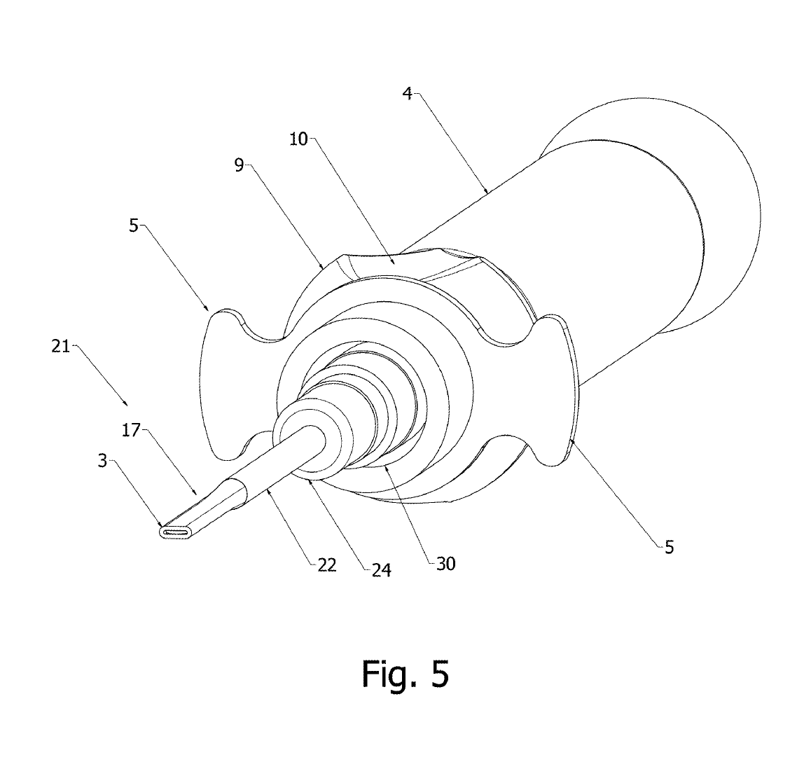

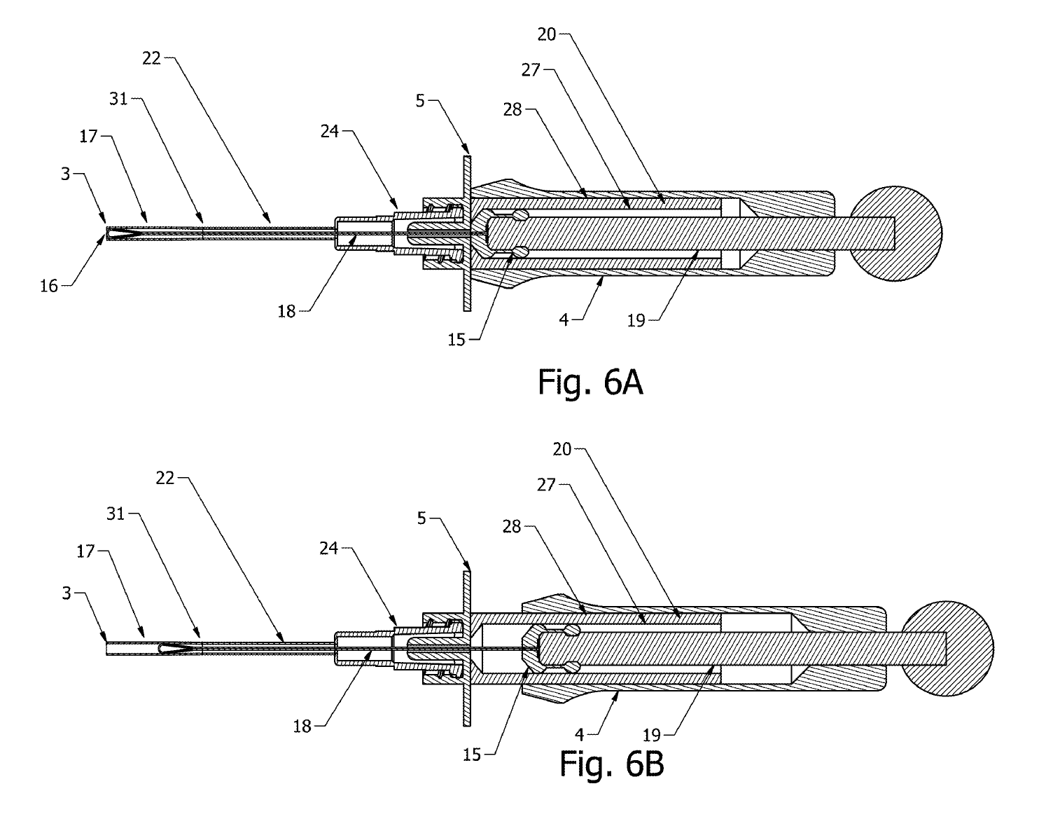

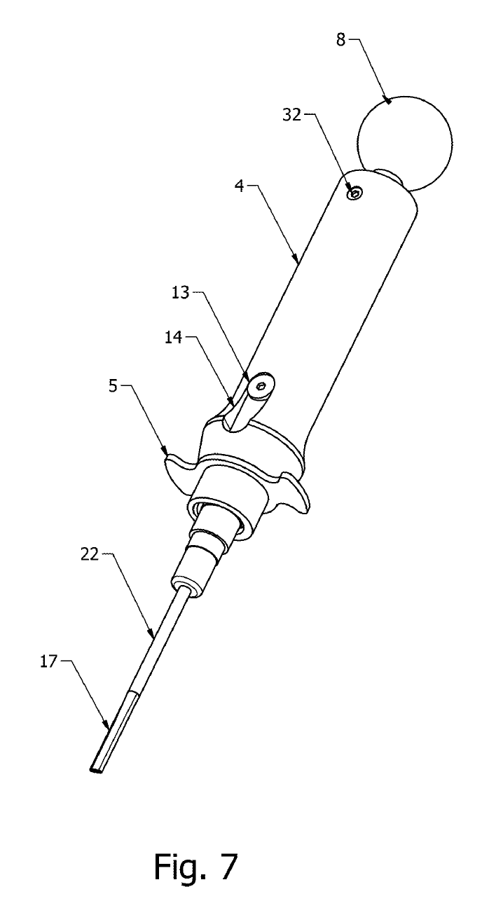

FIG. 3 and FIG. 7 depict views of opposing sides of a delivery device of the invention. FIG. 4A depicts an exploded of the device. FIG. 4B depicts an exploded view of the guide of the device. FIG. 4C depicts an exploded view of the discharge unit. FIG. 5 depicts a magnified perspective view. FIG. 6A and FIG. 6B depict sectioned elevation views of the device in the discharged and loaded state, respectively.

As in Example 1, the device comprises a discharge unit 26 comprising a mandrel 1 and a sleeve 4, and further comprises a guide 2 comprising a body 20 and nozzle 17 having an open distal end 3 for discharging a loaded implant (implant not shown). The mandrel 1 is disposed internally to the guide 2 and is configured to move relative to the guide 2 to discharge a loaded implant from the open end 3. The sleeve 4 is disposed externally to the body 20 of the guide 2 and is attached to the proximal end of the mandrel 1 for longitudinal movement therewith relative to the guide 2 (i.e. advancement or retraction relative to the open distal end 3).

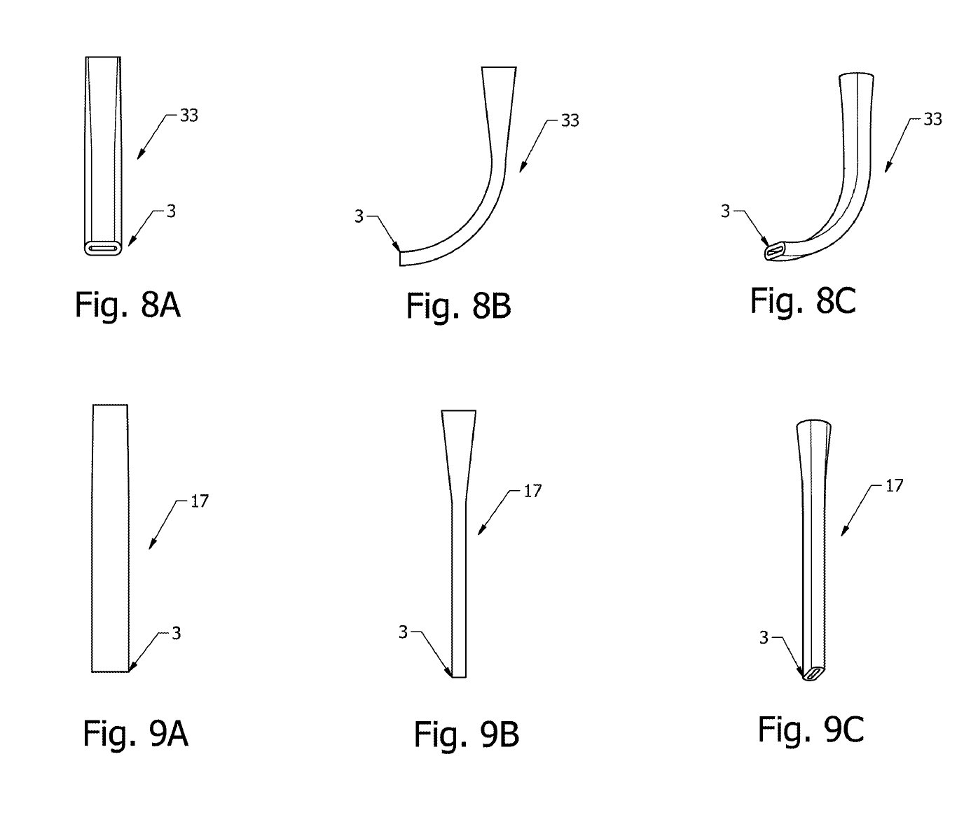

The body 20 of the guide 2 is constructed from a syringe barrel having a luer fitting 30 at its distal end. Coupled to the body 20 is a distal guide segment 21 comprising a nozzle 17, a segment proximal of the nozzle 22, and luer fitting 24. The nozzle 17 is constructed from a cylindrical FEP tube that has been flattened the distal end 3 to near the proximal end 31 of the nozzle 17 to provide a wide nozzle, e.g. as can be seen in FIG. 5 and FIG. 10. As an alternative to a wide nozzle, the nozzle is, for example, cylindrical along its entire length or has a distal end with a circular cross section (not shown). The proximal end 31 of the nozzle 17 is cylindrical and is attached with adhesive the segment proximal of the nozzle 22, which is a cylindrical cannula constructed from syringe needle having a blunt tip. The proximal end 31 of the nozzle has the same diameter as the segment proximal of the nozzle 22. The luer fitting 24 of the distal guide segment 21 is coupled to the luer fitting 30 of the body 20 to provide a functioning guide. The nozzle 17 is optionally straight, as depicted in FIG. 3 and FIG. 9A-C, or curved as depicted in FIG. 17 and FIG. 8A-C. When the nozzle is curved, the mandrel is optionally flexible relative to the nozzle such that the mandrel conforms or becomes curved as is travels through the nozzle. While the nozzle itself can have flexibility such that it deforms or curves when pressed against tissue, a curved nozzle allows the user to easily angle the distal end of the nozzle relative to the portion of the guide that transverses the insertion site, e.g. to slide under the retina into the subretinal space after insertion through the pars plana of the eye.

The mandrel 1 comprises a pushrod 18 made from a steel wire looped at the distal end 16 to provide a wide tip pushrod and further comprises a plunger arm 19 as a segment proximal of the pushrod 18. Constructed from a steel wire, the pushrod 18 is thin, flexible and relatively fragile while the plunger arm 19 is has a substantially greater diameter, is rigid or semi-rigid, and provides itself as a resilient member for attachment to the sleeve 4, e.g. by set screw 32. A plunger head 15 is disposed intermediately of the pushrod 18 and the plunger arm 19. The plunger head 15 is constructed from a thermoplastic elastomer provides a snug, leak-free fit in the body 2 and acts as a fluid pump as it moves longitudinally through the body 2. In this configuration, the plunger head 15 is configured to impart suction at the open end 3 of the guide by pumping fluid. Suction at the open end 3 imparted by the plunger head 15 can be used to load an implant, i.e. by “sucking” an implant into the nozzle 3.

The device further comprises two finger tabs 5 extending laterally from the body 20 of the guide 2 in opposing directions. The finger tabs 5 each comprise a distal surface 7 configured for interfacing independent fingers situated about opposing surfaces of the guide 2.

The discharge unit 26 comprises a butt end 8 having a convex surface. As depicted, the butt end 8 is disposed at the proximal end of the discharge unit 26 and is provided by an acrylic ball having a hole 26 for acceptance of and attachment to the proximal end of the mandrel 1.

The sleeve 4 is a cylindrical shell having an open distal end for sliding over the proximal end of the body 2. As in Example 1, the diameter of the sleeve 4 is sized such that the luminal (i.e. inner) surface 28 of the sleeve 4 makes sliding contact with the outer surface of the body 20. The sleeve 4 comprises a lip 9 at the distal end and a cavity 10 in the lip 9. The lip 9 and cavity 10 provide an interface for the user's thumb. In the examplary configuration shown, the user can place his thumb on the proximal taper 12 of the lip 9 with the tip of his thumb resting in the cavity 10.

A rotation limiter and retraction limiter is constructed from a pin 13 and a longitudinal slot 14. The pin 13 extends laterally from the body 20 and the longitudinal slot 14 is provided in the sleeve 4. The longitudinal slot 14 accepts the pin 13 and provides a pin track through which the pin 13 is free to move longitudinally from the distal end of the slot 14 to the proximal end of the slot 14 and is prevented from moving circumferentially, thus preventing rotation of the body 20 relative to the sleeve 4, which in turn prevents rotation of the mandrel 1 in the nozzle 17. Prevention of rotation of the mandrel within the nozzle ensures that the mandrel is maintained in a desired orientation and, in delivery devices having, for example, a wide nozzle and a delicate wide tip mandrel (e.g. formed from a looped wire) housed within, prevents damage to the wide tip mandrel from the nozzle.

In the discharged state (FIG. 6A), the distal end 16 of the mandrel 1 is flush with the open distal end of the nozzle. In the loaded state (FIG. 6B), the distal end 16 of the mandrel 1 is retracted relative to the nozzle less than an amount that would retract the distal end 16 of the mandrel 1 from the nozzle 17 (i.e. less than retracted back to the proximal end 31 of the nozzle 17 just distal of the segment proximal the nozzle 22). The distance retracted can be set by the user and/or limited by a retraction limiter, e.g. determined by the length of the slot 14.

As in Example 1, the luminal surface 28 of the sleeve 4 makes sliding contact with the body 20 about a large and elongated surface area, thereby providing smoothly guided longitudinal movement of the discharge unit 26 relative to the guide 2. The sleeve 4 is configured such that the surface area of sliding contact between the body 20 and the luminal surface 28 of the sleeve 4 increases as the sleeve 4 is advanced relative to the body 20. As can be seen by comparing the longitudinal position of the mandrel distal end 16 relative to the open distal end of the nozzle 3 in the loaded state (FIG. 6B) and the discharged state (FIG. 6A), the longitudinal length of the elongated surface area that makes sliding contact between the sleeve 4 and the body 20 is substantially greater (e.g. at least three times greater) than the distance of longitudinal movement required to discharge a loaded implant. As can be further seen, the contact diameter of the body 20 and sleeve 4 (i.e. outer diameter of the body and inner diameter of the sleeve which form the surface area making sliding contact) is substantially greater (e.g. at least 2 time greater or at least 5 times greater) than the outer diameter of the nozzle 17. Independently or combined, the length and diameter of the sliding contact surface area between the sleeve 4 and the body 20 provides an extremely smooth and guided longitudinal movement of the discharge unit 26 relative to the guide 2.

The device is configured (i.e. sized and shaped) such that the butt end 8 can be abutted against the palm of a user's hand while the user curls his forefinger and middle finger over the finger tabs 5 to interface the distal surfaces 7 and places his thumb on the lip 9 and in the cavity 10. In this orientation, discharge of the implant occurs upon the user pulling his fingers towards the proximal end of the device to retract the guide 2 relative to the mandrel. The butt end 8 can be abutted against the palm and the thumb on lip 9/cavity 10 act independently or in concert to stabilize the discharge unit 26 while the guide 2 is retracted by the finger tabs 5. Accordingly, this device allows the user to discharge the implant via injection or ejection, wherein injection comprises stabilizing the finger tabs 5 and advancing the discharge unit 26 (e.g. by applying a force to the butt end 8) as depicted in FIG. 12, and wherein ejection comprises stabilizing the discharge unit 26 (e.g. by interfacing the sleeve 4 or the butt end 8) and retracting the guide 2 (e.g. by applying a force to the finger tabs 5) as depicted in FIG. 11.

The nozzle is optionally configured with the dimensions of any of the examples listed in Table 2. Additionally or alternatively, the device is optionally configured with the dimensions of any of the examples listed in Table 2 and shown in FIG. 20A-C.

### Example 3

### Preparation of an Implant—Epoxy Based Viscous Polymer Layer Containing RPE Cells

An implant was prepared for implantation to the subretinal space using a device of the present invention, e.g. a device as detailed in Example 2. As a non-limiting example, the implant can comprise 8 micron thick layer of epoxy-based viscous polymer (e.g. SU-8) having pores with a diameter of about 5 microns and further comprising RPE cells. The layer of epoxy based viscous polymer is formed, e.g. by photolithography.

The cells can be made by any method. For example, RPE cells can be made, e.g. by spontaneous differentiation or by a method taught by Singh et al. (“Functional Analysis of Serially Expanded Human iPS Cell-Derived RPE Cultures” IOVS, October 2013, Vol. 54, No. 10, pp 6767-6778).

### Example 4

### Preparation of an Implant—Sheet Containing RPE Cells

An implant was prepared for implantation to the subretinal space using a device of the present invention, e.g. a device as detailed in Example 2. Although any method of preparing a substance may be used, the following method was used to prepare RPE cells.

RPE cells were harvested as sheets using dispase as described previously [Tezel T H, Del Priore L V, Kaplan H J. Harvest and storage of adult human retinal pigment epithelial sheets. Current eye research 1997; 16:802-809]. In brief, following removal of the sclera, eyes were treated with 2% dispase for 40 minutes at 37 degrees C. Dispase-treated eyes were washed in DMEM and an incision was made into the subretinal space. RPE sheets were isolated, gently placed on Transwell membrane insert [Corning Life Sciences, Wilkes Barre, Pa.], and were cultured in growth media consisting of DMEM supplemented 10% FBS, 100 IU/ml penicillin, and 100 g/ml streptomycin. Monolayer cell sheets were harvested one week later for retinal implantation.

Additionally or alternatively, RPE cells can be made, e.g. by spontaneous differentiation or by a method taught by Singh et al. (“Functional Analysis of Serially Expanded Human iPS Cell-Derived RPE Cultures” IOVS, October 2013, Vol. 54, No. 10, pp 6767-6778).

### Example 5

### Delivery of an Implant to the Retina

Devices according to Example 2 are tailored for delivery of an implant comprising RPE cells (e.g. prepared according to Example 3 or Example 4) to the retinas of three subject—a human, a rat, and a horse which have eye diameters of 24 mm, 6.5 mm, and 34 mm, respectively. Each device is identical except for the selected nozzle.

The device used to deliver an implant to the human has a curved nozzle (e.g. as depicted in FIG. 8A-C and FIG. 17) with a wide tip. The guide has an insertable length (Dimension B shown in FIG. 20C) of 26 mm and the distance from the distal end of the nozzle to the finger tabs (Dimension A shown in FIG. 20C) is 51 mm. The diameter of the nozzle is no greater than 2 mm. For example the width of the nozzle can be 1 mm and the height of the nozzle is substantially less than 1 mm. While holding the device in the user's hand, the hand is optionally rested on the subjects head while the nozzle is positioned at the target site (retina).

The device used to deliver an implant to the rat has a straight nozzle (e.g. as depicted in FIG. 9A-C and FIG. 3) with a wide tip or is a cylindrical nozzle. The guide has an insertable length (Dimension B shown in FIG. 20A) of 19 mm and the distance from the distal end of the nozzle to the finger tabs (Dimension A shown in FIG. 20A) is 45 mm. The diameter of the nozzle is no greater than 0.8 mm. For example the width of the nozzle can be 0.5 mm. The rat is laid on an operating table and, while holding the device in the user's hand, the hand is optionally rested on the operating table while the nozzle is positioned at the target site (retina).

The device used to deliver an implant to the horse has a curved nozzle (e.g. as depicted in FIG. 8A-C and FIG. 17) with a wide tip. The guide has an insertable length (Dimension B shown in FIG. 20C) of 38 mm and the distance from the distal end of the nozzle to the finger tabs (Dimension A shown in FIG. 20C) is 64 mm. The diameter of the nozzle is no greater than 2.5 mm. For example the width of the nozzle can be 1.5 mm and the height of the nozzle is substantially less than 1.5 mm. While holding the device in the user's hand, the hand is optionally rested on the subjects head while the nozzle is positioned at the target site (retina).

Specifically, the methods of delivery comprises loading an implant into the guide, inserting the guide into an eye, inserting the open end of the nozzle in the subretinal space in the eye, and advancing the mandrel relative to the guide to discharge the implant at the target site. Optionally, the method comprises making an incision in the eye before inserting the mandrel guide through the incision. Optionally, the method comprises inserting the mandrel guide through the pars plana in the region of the ciliary body at the periphery of the retina (e.g. using a curved nozzle) or inserting the mandrel through the sclera (e.g. using a cylindrical nozzle). Optionally, the method comprises incising the retina near the diseased target site giving access to the subretinal space.

### Example 6

### Guide with Side Port

A guide useful in the present invention optionally comprises a port in a sidewall thereof (‘side port’). Such a side port can be used to load an implant into the delivery device. FIG. 21A depicts an example of a useful distal guide segment 210 comprising a side port 403. FIG. 21B depicts an exploded view of the distal guide segment 210. It comprises a nozzle 402 and a segment proximal of the nozzle 404. Optionally, the segment proximal of the nozzle 404 is a syringe needle (e.g. blunt syringe needle) such as a 16 gauge needle. For illustration purposes, the segment proximal of the nozzle 404 is hereinafter referred to as the needle.

Optionally, the distal guide segment 210 is provided by inserting a proximal segment extending from the nozzle 402 (e.g. proximal end 311) into the distal end 310 of the needle 404. The proximal segment extending from the nozzle 402 is tubular from the proximal end 311 to the open distal end 300 for massage of a mandrel there through. The proximal segment can comprise a side port 4030 and can be inserted into the needle 404 such that the side port 4030 is aligned with side port 403 to form the distal guide segment 210. The nozzle 402 and the tubular proximal segment extending from the nozzle 402 (which comprises side port 4030) can optionally be provided as a single molded or extruded product (e.g. plastic such as FEP), or by other manufacturing process that provides a seamless construction. As depicted, the guide can have a bilayer (or other multilayer) construction such that a first layer (e.g. nozzle 402 and the tubular proximal segment extending from the nozzle 402) is disposed within a second layer (e.g. needle). Such a device can be optionally be configured such that the first layer provides a seamless path from the port to the distal end and the second layer (e.g. needle) provides rigidity and/or structural integrity to the first layer. For example, the first layer including the nozzle may be constructed from a delicate and/or flexible material to target delivery to a sensitive target site (e.g. in an an eye). Such a first layer can be then be constructed in an extremely delicate and/or flexible material that would otherwise be weakened by the introduction of a port 4030 in its side wall (e.g. prone to buckling due to the port removing sidewall material), but which is supported by the second layer having respective port 403 that aligns with port 4030. Another advantage of a bilayer construction is that formation of the guide by inserting first layer into a second layer, e.g. with or without a side port or seamless construction, reduces manufacturing burdens and is easily reproducible on a large scale.

The distal guide segment 210 depicted FIG. 21A, can be configured such that an implant can be loaded through the side port 403, e.g. by retracting a mandrel (not shown) to a position proximal the side port 403 and then advancing the mandrel to deliver the implant by discharging the implant from open distal end 300 of the nozzle 402.

In the present invention, a side port allows side-loading of implants, e.g. as opposed to front loading via suction at the distal end of the nozzle. Among other optional advantages, such side-loading can surprisingly allow precise orientation of any implant in the guide, e.g. by visually guided manual placement. Such a feature is surprisingly advantageous for any implant, and further advantageous for implants comprising a plurality of stacked or adjacent members (e.g. membranes) that can be precisely placed in a desired position relative to each other in the guide.

The distal guide segment can comprise a Luer fitting 24, e.g. for connection to an optional Luer fitting 30 of a guide body of a delivery device of the invention. Alternatively, the distal guide segment can be connected to a guide body of a delivery device of the invention in any other manner.

### Example 7

### Laterally Deformable Distal Member

A delivery device of the present invention optionally comprises a pushrod having, at the distal end, a member (‘distal member’), that expands and/or compresses laterally (is ‘laterally deformable’). Such a laterally deformable distal member can optionally be configured to have a snug fit in the lumen of the guide (e.g. completely filling a cross-sectional area of the lumen) and, for example, provide a seal behind a loaded implant.

For example, FIG. 25 depicts a distal member 401 in two positions, retracted and advanced, inside a nozzle 402, each position exhibiting a different state of lateral deformation. The nozzle 402 is tapered from a proximal portion (right), having a relatively larger inner diameter, down to a distal portion (left), having a relatively smaller inner diameter. When in the retracted position (shown in solid lines on right), the distal member 401 has a diameter that matches the lumen (i.e. inner diameter) of the larger proximal portion of the nozzle 402. When in the advanced position (shown in dashed lines on left), the distal member 401 has a diameter that matches the lumen (i.e. inner diameter) of the smaller distal portion of the nozzle 402.

Such a distal member expands and contracts to completely fill the cross-sectional area of the lumen of a guide or nozzle thereof. Optionally, such a distal member expands and contracts to completely fill the cross-sectional area of the lumen in a plurality of portions of a guide or nozzle, including a plurality of portions with different inner diameters, e.g. as in a tapered guide or nozzle thereof. This feature can be ensure a seal is always present behind the implant and/or maximize the surface area and potential implant/pushrod contact area in the lumen of the guide. Such a feature is advantageous for any implant, e.g. a implant comprising a plurality of stacked or adjacent substances (e.g. membranes).

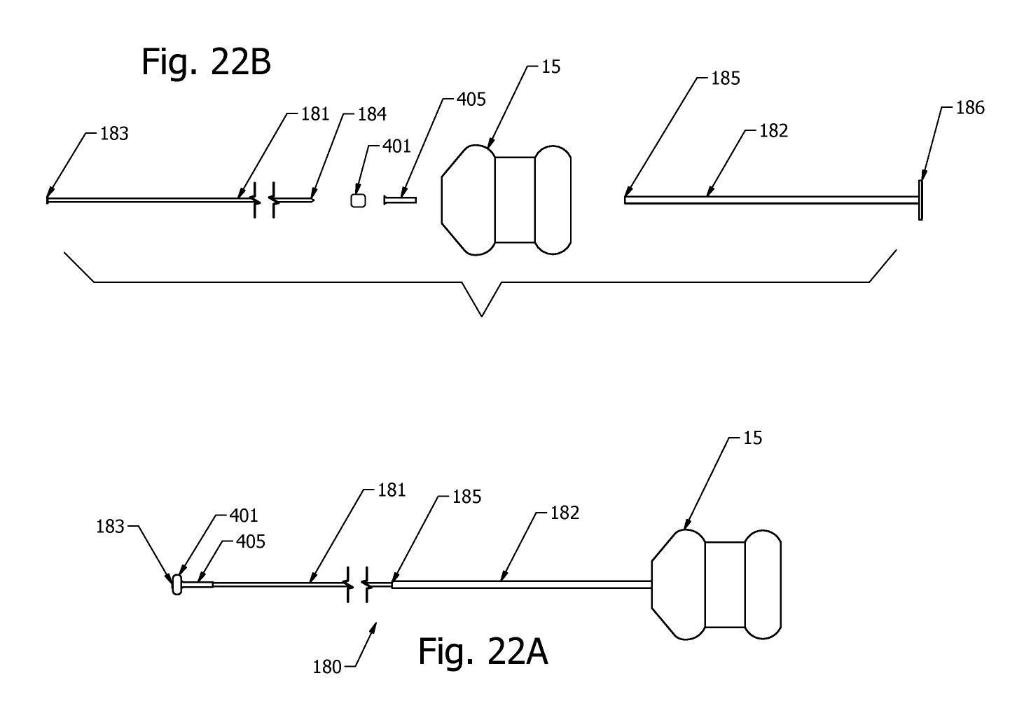

Additionally or alternatively, the laterally deformable distal member can be configured as shown in FIG. 24. FIG. 24 depicts a distal member 401 in different states of deformation: a resting state (left) and a laterally expanded state (right). In FIG. 24, the laterally expanded state optionally imparted by longitudinally compressing the distal member 401, e.g. by applying opposing longitudinal forces on the distal member 401 from a compression sleeve 405 against a swaged end 183 of a rod 180 after inserting the rod 180 through the distal member 401 (e.g. as depicted in FIGS. 22A and 22B). Such a distal member can be expanded as needed, e.g. to provide to provide a sliding fit or snug fit in the lumen of a guide or nozzle thereof, e.g. as depicted in FIG. 25). Among other advantages, the present inventors have surprisingly discovered that such a compression-expanded (i.e. expanded laterally via longitudinal compression) distal member can be laterally compressed (e.g. upon traveling through a tapered nozzle segment having a reduced diameter), then laterally expanded (e.g. upon exiting the distal end of a nozzle) and re-compressed laterally without damaging the distal member (e.g. by retracting, back into the nozzle, a distal member that has laterally expanded after exiting the distal end of the nozzle).

### Example 8

### Pushrod having a Laterally Deformable Distal Member

A delivery device of the present invention optionally comprises a pushrod comprising a laterally deformable distal member, e.g. as detailed in Example 7. The pushrod can be configured as depicted in FIG. 22A, which depicts a pushrod 180 connected to a plunger 15. An exploded view is depicted in FIG. 22B. The pushrod 180 comprises, at its distal end, a laterally deformable member 401 for interfacing (e.g. pushing and ejecting) an implant. The laterally deformable member 401 can optionally be expanded laterally with a longitudinal compressive force such as by sandwiching the laterally deformable member 401 between two fixed (or fixable) members. For example, pushrod can comprise a distal rod 181 having a swaged distal end 183 against which the distal member 401 is longitudinally compressed by a compression sleeve 405, thus laterally expanding the distal member 401.

An optional manufacturing method includes steps of inserting the proximal end 184 of the distal rod 181 through the distal member 401 and then through the compression sleeve, longitudinally compressing the distal member 401 between the swaged distal end 183 and the compression sleeve 405, and then fixing the compression sleeve 405 to the distal rod 183 (e.g. by weld or adhesive). The distal rod is then optionally inserted through a plunger 15 and then fixed to a proximal rod 182 (e.g. by weld or adhesive). For example, proximal rod 182 can be a tube (e.g. 26 gauge tube) through which the proximal end 184 of the distal rod 181 (e.g. 0.008″ wire) is inserted. The proximal rod 182 can optionally have a stop such as disk 186 configured to prevent longitudinal movement of the pushrod 180 relative to the plunger.

### Example 9

### Delivery Device

A delivery device of the present invention is provided comprising one or both of a guide with a side port, as detailed in Example 6, and a pushrod having a laterally deformable distal member, as detailed in Example 8.

As one example, a delivery device is provided as depicted in detailed in FIG. 23A through FIG. 23E. Specifically, the delivery device is the same as the delivery device detailed in Example 2 (e.g. as depicted in FIG. 3 through FIG. 7), except that the distal guide segment 21 (e.g. depicted in FIG. 4B) is replaced with a distal guide segment having a side port (e.g. distal guide segment 210 depicted in FIG. 21A), and the pushrod 18 (e.g. depicted in FIG. 4C) is replaced with a pushrod having a laterally deformable distal member (e.g. pushrod 180 depicted in FIG. 22A).

The citations provided herein are hereby incorporated by reference for the cited subject matter.

1. A device comprising:
a. a guide, wherein the guide has an open distal end configured for discharging an implant when loaded therein; and
b. a discharge unit, wherein
i. the discharge unit comprises a mandrel and a sleeve connected to each other that move together relative to the guide;
ii. the mandrel is disposed internally to the guide, whereby relative movement of the mandrel through the guide towards the open distal end biases the loaded implant to move longitudinally towards the open distal end; and
iii. the sleeve is disposed externally to the guide.

2. The device of claim 1, wherein the guide comprises at least one finger tab extending laterally therefrom.

3. The device of claim 2, wherein the at least one finger tab extends laterally from the guide in opposing directions.

4. The device of claim 2, wherein the at least one finger tab is disposed distally of the sleeve.

5. The device of claim 2, wherein the discharge unit comprises a proximal surface, and wherein device is configured for:
a. abutting the proximal surface of the discharge unit against a palm of a user's hand; and
b. presenting the at least one finger tab for interfacing with at least one finger of said palm, whereby movement of the at least one finger towards said palm imparts said movement of the mandrel through the guide.

6. The device of claim 5, wherein:
a. the at least one finger tab comprises a distal surface configured for interfacing said at least one finger; and
b. said imparting movement of the mandrel through the guide comprises applying a normal force to the distal surface of at least one finger tab towards said palm.

7. The device of claim 5, wherein the device comprises a convex surface on the proximal surface of the discharge unit.

8. The device of claim 2, wherein the device is configured for both an injection mode and an ejection mode, wherein the injection mode comprises stabilizing the at least one finger tab and advancing the discharge unit towards the distal end, and wherein the ejection mode comprises stabilizing the discharge unit and retracting the at least one finger tab towards the proximal end of the discharge unit.

9. The device of claim 1, wherein the sleeve comprises a finger grip, optionally wherein the finger grip comprises one or more of a cavity and a lip, optionally wherein the lip is tapered.

10. The device of claim 9, wherein the sleeve comprises a lip and a cavity, wherein the cavity is in the lip.

11. The device of claim 1, further comprising a mechanism configured for limiting rotation of the mandrel relative to the guide (‘rotation-limiter).

12. The device of claim 11, wherein the rotation limiter is provided by a first member comprised by the guide and a second member comprised by the discharge unit, wherein the first member and the second member are disposed circumferentially of each other and configured to move longitudinally relative to each other.

13. The device claim 12, wherein the first member is a pin extending laterally from the guide and the second member is a pin track comprised by the sleeve about which the pin moves longitudinally, optionally wherein the pin is recessed in the sleeve.

14. The device of claim 13, wherein the pin track is a slot or a groove.

15. The device of claim 1, further comprising a mechanism for limiting retraction of the mandrel relative to the guide (‘retraction limiter’).

16. The device of claim 1, wherein the mandrel comprises a plunger head disposed proximally of the distal end of the mandrel, wherein the plunger head has a snug-fit within the guide.

17. The device of claim 16, wherein the plunger head is configured to impart suction at the open end of the guide upon retraction of the mandrel relative to the guide.

18. The device of claim 1, wherein the guide comprises a curved or flexible nozzle and the mandrel comprises a pushrod and a proximal segment disposed proximally of the pushrod, wherein the pushrod is flexible relative to the proximal segment.

19. The device of claim 18, wherein the mandrel comprises a plunger head disposed intermediately of the pushrod and the proximal segment, optionally wherein the plunger head has a diameter greater than that of proximal segment and the pushrod.

20. The device of claim 1, wherein the guide comprises a removable nozzle.

21-88. (canceled)
